(19) United States
(12) Patent Application Publication
(10) Pub. No.: US 2026/0193634 A1
(43) Pub. Date: Jul. 9, 2026

(54) System For Modulating Receptor Tyrosine Kinase Signaling And Methods Of Use Thereof

(71) Applicant: THE JOHNS HOPKINS UNIVERSITY, Baltimore, MD (US)

(72) Inventors: Michael Jerry Caterina, Baltimore, MD (US); Stephen John Gould, Baltimore, MD (US); Shang-Jui Tsai, Baltimore, MD (US)

(21) Appl. No.: 19/128,151

(22) PCT Filed: Nov. 9, 2023
(86) PCT No.: PCT/US2023/037083

§ 371 Date: May 7, 2025

Related U.S. Application Data
(60) Provisional application No. 63/424,414, filed on Nov. 10, 2022.

(51) Int. Cl. C12N 9/50; A61K 38/48; A61K 48/00; A61P 29/00; C07K 14/435

(52) U.S. Cl. CPC C12N 9/506; A61K 38/4873; A61K 48/005; A61P 29/00; C07K 14/43504; C12Y 304/22044; C07K 2319/00

(57) ABSTRACT

Provided herein is an engineered controllable membrane targeting system based on triggered exposure of a cryptic acylation signal, a calcium-regulated protease that can be used to expose this cryptic acylation signal, a means of coupling transmembrane receptor signaling events to the activation of target acylation through this and other protease-dependent systems, and methods of use thereof. The use of this system recruits signal modulators to the plasma/endosomal membranes in response to transmembrane signaling events. The system includes an expression vector including a pro-effector protein module and a protease module. The system can be used for modulating a receptor tyrosine kinase (RTK) signaling, and for treating a subject having chronic pain, diabetes, an inflammatory disorder, cancer, or disease caused by hyperactive cell signaling pathway.

### CROSS-REFERENCE TO RELATED APPLICATIONS

This application claims the benefit of priority under 35 U.S.C. § 119(e) of U.S. Provisional Application No. 63/424,414, filed Nov. 10, 2022. The disclosure of the prior application is considered part of and is herein incorporated by reference in the disclosure of this application in its entirety.

### STATEMENT REGARDING GOVERNMENT FUNDING

This invention was made with government support under Grant No. R21N128599 awarded by the National Institutes of Health. The government has certain right in the invention.

### BACKGROUND OF THE INVENTION

### Field of the Invention

The present invention relates generally to receptor tyrosine kinase (RTK) signaling modulation, and more specifically to conditional modulation of RTK signaling to achieve anti-hyperalgesic outcome.

### Background Information

Many diseases involve the hyperactivity of receptor tyrosine kinase signaling. A prime example of such a disease is chronic pain. While chronic pain can arise from many etiologies, one common feature is that there are often increases in the activities of “pro-nociceptive” signal transduction pathways that promote the excitability of specialized sensory neurons called nociceptors. These pathways can include activation of receptor tyrosine kinases (RTKs, e.g., the Nerve Growth Factor Receptor, TrkA) or activation of G protein-coupled receptors (GPCRs, e.g., receptors for prostaglandins) that in turn activate Gq and Gs proteins. Many pharmacological treatments for pain are focused either on disrupting events upstream of these pro-hyperalgesic signaling pathways (e.g., aspirin, ibuprofen) or engaging endogenous “anti-hyperalgesic” signaling pathways such as those activated by Gi-coupled opiate receptors (e.g., morphine and its derivatives). Current therapies for pain are inadequate, however, either due to limited efficacy, challenges with dosing or timing, or unwanted side effects. One potential approach to circumventing these limitations would be the development of a therapeutic strategy in which endogenous pathological signaling through RTKs could be used as the “input” to dictate the activation of an “output” molecule that could block signaling from RTKs, Gq and/or Gs-coupled GPCRs and/or stimulate signaling downstream of Gi-coupled GPCRs. A shared feature of many of these pro- and anti-nociceptive signaling pathways is the participation of guanine nucleotide triphosphate hydrolases (GTPases). Many of the relevant GTPases are localized at the plasma or endosomal membranes. Based on these considerations, the present disclosure describes a system allowing conditional delivery of either GTPase inhibitors or GTPase activators to the plasma membrane (and possibly endosomal membrane) compartments of the cell in response to RTK signaling to “short-circuit” RTK signaling, which would normally be pro-hyperalgesic, to achieve an anti-hyperalgesic outcome.

Pathological pain results in part from a disequilibrium between pro- and anti-hyperalgesic signaling pathways in nociceptive neurons. Many available pain therapies are designed to inhibit specific pro-hyperalgesic signals or enhance specific anti-hyperalgesic signals. However, an alternative approach would be to proportionately redirect endogenous pathological pro-hyperalgesic signaling towards activation of anti-hyperalgesic pathways. Such an approach could clamp nociceptive function under evolving pathological conditions and do so without compromising protective pain reflexes. Provided herein is a strategy to redirect signaling from the pro-hyperalgesic TrkA receptor to achieve inhibition of pro-hyperalgesic cAMP and Ras/Rap driven signaling.

### SUMMARY OF THE INVENTION

The present invention is based on the seminal discovery of a modular and customizable system, referred as Inducible Membrane Anchoring (IMA), that operates on the principle that many effector proteins function most efficiently when recruited to the plasma membrane, where their targets reside to redirect signaling from the pro-hyperalgesic TrkA receptor to achieve inhibition of pro-hyperalgesic cAMP and Ras/Rap driven signaling.

In one embodiment, the present invention provides an expression vector including: (a) a nucleic acid sequence encoding a pro-effector protein, and (b) a nucleic acid sequence encoding a protease, wherein the pro-effector protein is a fusion protein comprising of a spacer protein, a protease substrate, a peptide substrate for post-translational acylation, and an effector protein, and wherein the protease of interest is a fusion protein containing a protease.

In one aspect, the nucleic acid sequences are operably linked to an expression control sequence. In some aspects, the expression vector includes pS622, pS458, pS459, pS462, pS463, pS485, pS498, pS492, pS634, pS577, pS579, pS630, pS631. In one aspect, the spacer protein is a single- or a multi-component, including one or more of (i) a fluorescent protein, (ii) a SNAP Tag, (iii) a HaloTag, (iv) an epitope tag, (v) a dimerization domain for kinase substrates, (vi) a binding domain for kinase substrates, (vii) a peptide substrate for kinases, (viii) a proteolysis-sensitive destabilization domain or (ix) a combination thereof. In some aspects, the dimerization domain is selected from the group consisting of a FK506-binding protein, a Gibberellin receptor 1, and an abscisic acid receptor ABI1. In other aspects, the binding domain for kinase substrates is selected from the group consisting of a Src-homology 2 domain, a phosphotyrosine binding domain, a Forkhead-associated 1, and a WW domain. in some aspects, the proteolysis-sensitive destabilization domain is derived from mouse ornithine decarboxylase. In another aspect, the protease substrate is suitable for virus-derived proteases or non-virus-derived proteases. In some aspects, the peptide substrate for post-translational acylation is a substrate of methionine aminopeptidase and/or N-myristoyltransferase. In one aspect, the effector protein includes one or more components of (i) a Ras-binding domain derived from c-Raf or mutants thereof, (ii) a Gq alpha subunit inhibitor derived from phospholipase C beta 3 or mutants thereof, (iii) an adenylyl cyclase inhibitor derived from Gi alpha subunits or mutants thereof, (iv) a voltage-gated calcium channel inhibitor derived from protein Rem1 or mutants thereof, (v) any other effector domain, (vi) a fluorescent protein, (vii) a SNAP Tag, a HaloTag, (viii) an epitope tag, (ix) a protein domain for chemically induced dimerization or (x) any combination thereof. In some aspects, the protein domain for chemically induced dimerization is selected from the group consisting of FK506-binding protein, Gibberellin receptor 1, and abscisic acid receptor ABI1. In other aspects, the protease includes a plant virus-derived protease. In some aspects, the plant virus-derived protease is a tobacco etch virus (TEV) protease, tobacco vein mottling virus (TVMV) protease, or any mutant thereof. In other aspects, the plant virus-derived protease is a modified form with an attached autoinhibitory domain, a modified form sensitive to calcium. in many aspects, the autoinhibitory domain is EYVRFAP. In one aspect, the fusion protein containing a protease further includes one or more components selected from the group consisting of a protein domain for chemically induced dimerization, a binding domain for kinase substrates, and a peptide substrate for kinases.

In another embodiment, the invention provides a method of treating chronic pain in a subject including administering to the subject any of the nucleic acid expression vectors described herein, wherein administering includes delivering the expression vector into cells of the subject, thereby treating chronic pain in the subject.

In one aspect, delivering includes using a virus-mediated delivery method or non-viral delivery method. In some aspects, virus-mediated delivery method includes lentivirus, adeno-associated virus, or herpes simplex virus. In other aspects, non-viral delivery method includes synthetic mRNA loaded into lipid nanoparticles, synthetic mRNA loaded into exosomes, purified proteins mixed with cell-penetrating peptide or materials, Cas9/CRISPR mediated genome editing, homologous recombination in stem cells, cell transfection, or electroporation. In one aspect, the chronic pain is induced by osteoarthritis and/or tissue inflammation.

In an additional embodiment, the invention provides a method of treating a subject comprising administering to the subject any of the nucleic acid expression vectors described herein, thereby treating the subject.

In one aspect, the subject has chronic pain, diabetes, an inflammatory disorder, cancer, or disease caused by hyperactive cell signaling pathway.

In a further embodiment, the invention provides a method of modulating a receptor tyrosine kinase (RTK) signaling in a cell including contacting the cell with the nucleic acid any of expression vectors described herein, thereby modulating the RTK signaling in the cell.

In one aspect, the RTK is selected from the group consisting of TrkA TrkB, TrkC, ret-c containing neurotrophin receptors, epidermal growth factor receptor (EGFR), insulin receptor, insulin-like growth factor receptor, vascular endothelial growth factor receptor, platelet-derived growth factor, fibroblast growth factor, T-cell receptor and B-cell receptor.

### BRIEF DESCRIPTION OF THE DRAWINGS

FIG. 1 is a schematic representation of the mechanistic 5-step IMA pathway. Plasmids: S485, S492, mTrkA (OriGene), S754, S758, S622.

FIG. 2 is a schematic representation of the structure of the pro-effector. Plasmids: S576, S579, S622, S758, mTrkA (OriGene).

FIG. 3 is a schematic representation of the structure of the protease. Plasmids: S622, S492, JM1242, mTrkA (OriGene), S1066, S917, JM1260, EGFR (OriGene) and ErbB2 (OriGene), IGF1R (Addgene).

FIGS. 4A-4D illustrate the mechanism of action of the pro-effector and protease. FIG. 4A is a schematic representation of a response to activated tropomyosin-related kinase A (TrkA). FIG. 4B is a schematic representation of a response to activated receptor tyrosine kinase. FIG. 4C is a schematic representation of a response to activated receptor tyrosine kinase. FIG. 4D is a schematic representation of a response to activated kinase. Plasmids: S462, S463, 5715, S722

FIGS. 5A-5B illustrate ^{TrkA}IMA-mediated translocation of proeffector to the plasma membrane. FIG. 5A shows immunofluorescence images showing that ^{TrkA}IMA induces membrane anchoring dependent on active TrkA. FIG. 5B is a graph illustrating the quantification of membrane enrichment.

FIGS. 6A-6C illustrate ^{TrkA}IMA-mediated inhibition of Ras-dependent MAP Kinase signaling. FIG. 6A shows immunofluorescence images showing altered Ras-dependent ERK activity. FIG. 6B is a graph illustrating the quantification of membrane enrichment. FIG. 6C is a graph illustrating ERK activity quantification. Plasmids: S1094, S1067

FIGS. 7A-7C illustrate IMA signaling preference among a subset of receptor tyrosine kinases in HEK cells. FIG. 7A is a schematic representation of the components used, with SH2 adaptor domains labeled. FIG. 7B shows immunofluorescence images illustrating IMA mediated plasma membrane mNG enrichment. FIG. 7C is a graph illustrating the quantification of normalized plasma membrane mNG enrichment. Plasmids: S843, S844.

FIGS. 8A-8C illustrate the chemical dimerization induced translocation of mNG to membrane via IMA. FIG. 8A illustrates that Rapalog induced dimerization of FRB and FKBP drives translocation. FIG. 8B illustrates that Gibberellic Acid derivative (GA3-AM) induced dimerization of GAI1 and GID1 drives translocation. FIG. 8C is a graph illustrating the quantification of mNG membrane enrichment. Plasmids: S1067, S1093.

FIG. 9 is a schematic representation of the design of a Ca2+-activated form of TVMV protease. Plasmids: S717, S463.

FIGS. 10A-10C illustrate IMA mediated translocation of mNG to plasma membrane driven by dual treatment with chemical dimerizer and elevated Ca2+. FIG. 10A is a diagram of engineered Ca2+-dependent TVMV protease. FIG. 10B is a schematic showing dependence of proeffector cleavage on both rapamycin and Ca2+. FIG. 10C is a graph illustrating plasma membrane enrichment of mNG signal in cells treated. A, ionophore A23187. TG, thapsigargin. ATP, adenosine triphosphate. Individual data points represent separate coverslips.

FIGS. 11A-11B illustrate the use of a “chemical plus Ca2+”-dependent IMA system to inhibit Gq signaling. FIG. 11A is a graph illustrating percent cells responding to ATP. FIG. 11B is a graph illustrating percent cells responding to carbachol.

FIGS. 12A-12B illustrate GA3-AM inducible IMA mediated translocation of mNeon Green to the plasma membrane of cultured mouse DRG neurons. FIG. 12A shows immunofluorescent images in the absence of treatment. FIG. 12B shows immunofluorescent images after GA3-AM treatment.

FIG. 13 shows immunofluorescent images of dual stimulus conditional induction of membrane translocation of IMA proeffector mNeon Green in cultured mouse DRG neuron.

### DETAILED DESCRIPTION OF THE INVENTION

The present invention is based on the seminal discovery of a modular and customizable system, referred as Inducible Membrane Anchoring (IMA), that operates on the principle that many effector proteins function most efficiently when recruited to the plasma membrane, where their targets reside to redirect signaling from the pro-hyperalgesic TrkA receptor to achieve inhibition of pro-hyperalgesic cAMP and Ras/Rap driven signaling.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The present invention is directed to methods of conditionally delivering effector proteins to plasma and endosomal membranes in response to tyrosine kinase signaling. To achieve delivery of effector proteins to the plasma membrane and endosome compartments of a cell under the control of endogenous signaling by receptor tyrosine kinases the system described herein is used. This system can be referred to by two interchangeable acronyms: 1) Membrane Anchoring Gated by Inducible Cleavage (MAGIC) or 2) Inducible Membrane Anchoring (IMA)

The specific means by which this goal is achieved is through the use of a system bearing two protein components: 1) a “protease” module that binds to activated RTKs and 2) a “carrier” module or “pro-effector” module that also bind to the same activated RTKs, and that contains a latent effector domain capable of inhibiting or activating target GTPases. The strength of the protease and the susceptibility of the proteolytic substrate site on the carrier are both tuned such that the carrier is cleaved inefficiently when the protease and carrier are distributed freely in the cytoplasm but is cleaved more efficiently once the two are brought into close proximity at the cytoplasmic domain of the activated RTK. Proteolytic cleavage of the carrier has two consequences. First, it liberates the “downstream” portion of the carrier, which contains the effector domain. Second, it exposes on that downstream portion a cryptic acylation site. Endogenous lipid transferases covalently attach myristoyl and/or palmitoyl lipid groups (i.e., acyl groups) onto the liberated polypeptide, causing the effector domain to become re-localized to the plasma and endosomal membrane compartments, and thus come into contact with its intended GTPase targets.

Accordingly, provided here are (i) an engineered controllable membrane targeting system based on triggered exposure of a cryptic acylation signal; (ii) a calcium-regulated protease that can be used to expose this cryptic acylation signal; and (iii) a means of coupling transmembrane receptor signaling events to the activation of target acylation through this and other protease-dependent systems. The use of this system has been demonstrated to recruit signal modulators to the plasma/endosomal membranes in response to transmembrane signaling events, and specific combinations of adaptor domains that differentially allow signaling from specific protein kinases to drive the unmasking the cryptic acylation domain of latent effector polypeptides by the engineered proteases have been identified.

In one embodiment, the present invention provides an expression vector including: (a) a nucleic acid sequence encoding a pro-effector protein, and (b) a nucleic acid sequence encoding a protease, wherein the pro-effector protein is a fusion protein comprising of a spacer protein, a protease substrate, a peptide substrate for post-translational acylation, and an effector protein, and wherein the protease of interest is a fusion protein containing a protease.

As used herein, the term “nucleic acid” or“oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e. transfection of, cells, in particular, in the form of RNA which can be prepared by in vitro transcription from a DNA template. The RNA can moreover be modified before application by stabilizing sequences, capping, and polyadenylation.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663.

The term “vector”, “expression vector”, or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as a gene encoding a protein of interest, one or more protein tags, functional domains and the like. Vectors suitable for use in preparation of proteins and/or protein conjugates include those selected from baculovirus, phage, plasmid, phagemid, cosmid, fosmid, bacterial artificial chromosome, viral DNA, P1-based artificial chromosome, yeast plasmid, and yeast artificial chromosome.

The system described herein can be useful with a variety of alternative “pro-effector” and protease protein combination. Provided herein are non-limiting examples of such combinations. As used herein, the “protease protein” can also be referred to as the “protease”, the “recruitable protease”, or as a module thereof, without any change in the meaning. The “pro-effector protein” can also be referred to as the “pro-effector”, or as a “module” thereof, without any change in the meaning.

For example, the utility of the system has been demonstrated for Tropomyosin Related Kinase A (TrkA)-activated inhibition of ERK signaling through the use of the following components:
A “recruitable-Protease” module containing a modified Tobacco Vein Mottling Virus (TVMV) protease conjugated to a dimeric Src-Homology 2 (SH2) domain from Phospholipase C gamma 1 to permit binding to TrkA in a TrkA activity-dependent manner; and

A “RBD Pro-Effector” module containing: a cryptic acylation site, a downstream Ras Binding Domain (RBD) derived from c-Raf, and an upstream polypeptide containing an SH2 domain from the protein SH2B1 that masks the cryptic acylation site prior to proteolysis.

The utility of the system in the specific example of TrkA-activated inhibition of cAMP signaling has been demonstrated through the use of the following components:
A “recruitable-Protease” module containing a modified TVMV protease conjugated to a dimeric SH2 domain from Phospholipase C gamma 1 to permit binding to TrkA in a TrkA activity-dependent manner; and
A “AGS1 Pro-Effector” module containing: a cryptic acylation site, a downstream Activator of G-Protein Signaling 1, which activates the inhibitory G protein Gi, and an upstream polypeptide containing an SH2 domain from the protein SH2B1 that masks the cryptic acylation site prior to proteolysis.

The utility of the system in the specific example of TrkA-activated inhibition of Gq G protein signaling is being investigated through the use of the following components:
“Recruitable-Protease” module containing a modified TVMV protease conjugated to a dimeric SH2 domain from Phospholipase C gamma 1 to permit binding to TrkA in a TrkA activity-dependent manner; and
“Gq inhibitor Pro-Effector” module containing: a cryptic acylation site, a downstream Gq inhibitor domain, and an upstream polypeptide containing an SH2 domain from the protein SH2B1 that masks the cryptic acylation site prior to proteolysis.

In one aspect, the nucleic acid sequences are operably linked to an expression control sequence.

In some aspects, the expression vector includes pS622, pS458, pS459, pS462, pS463, pS485, pS498, pS492, pS634, pS577, pS579, pS630, pS631.

In one aspect, the spacer protein is a single- or a multi-component, including one or more of (i) a fluorescent protein, (ii) a SNAP Tag, (iii) a HaloTag, (iv) an epitope tag, (v) a dimerization domain for kinase substrates, (vi) a binding domain for kinase substrates, (vii) a peptide substrate for kinases, (viii) a proteolysis-sensitive destabilization domain or (ix) a combination thereof.

In some aspects, the dimerization domain is selected from the group consisting of a FK506-binding protein, a Gibberellin receptor 1, and an abscisic acid receptor ABI1.

In other aspects, the binding domain for kinase substrates is selected from the group consisting of a Src-homology 2 domain, a phosphotyrosine binding domain, a Forkhead-associated 1, and a WW domain. in some aspects, the proteolysis-sensitive destabilization domain is derived from mouse ornithine decarboxylase.

In another aspect, the protease substrate is suitable for virus-derived proteases or non-virus-derived proteases.

In some aspects, the peptide substrate for post-translational acylation is a substrate of methionine aminopeptidase and/or N-myristoyltransferase.

In one aspect, the effector protein includes one or more components of (i) a Ras-binding domain derived from c-Raf or mutants thereof, (ii) a Gq alpha subunit inhibitor derived from phospholipase C beta 3 or mutants thereof, (iii) an adenylyl cyclase inhibitor derived from Gi alpha subunits or mutants thereof, (iv) a voltage-gated calcium channel inhibitor derived from protein Rem1 or mutants thereof, (v) any other effector domain, (vi) a fluorescent protein, (vii) a SNAP Tag, a HaloTag, (viii) an epitope tag, (ix) a protein domain for chemically induced dimerization or (x) any combination thereof.

In some aspects, the protein domain for chemically induced dimerization is selected from the group consisting of FK506-binding protein, Gibberellin receptor 1, and abscisic acid receptor ABI1. In other aspects, the protease includes a plant virus-derived protease.

In some aspects, the plant virus-derived protease is a tobacco etch virus (TEV) protease, tobacco vein mottling virus (TVMV) protease, or any mutant thereof. In other aspects, the plant virus-derived protease is a modified form with an attached autoinhibitory domain, a modified form sensitive to calcium. In many aspects, the autoinhibitory domain is EYVRFAP.

In some examples, the plant virus-derived protease is a modified form sensitive to calcium, in which a module of a split-calmodulin domain surrounding a calmodulin substrate is inserted into the 61HisGlyc62 position of TEV protease or the 64HisGly65 position of TVMV protease.

In one aspect, the fusion protein containing a protease further includes one or more components selected from the group consisting of a protein domain for chemically induced dimerization, a binding domain for kinase substrates, and a peptide substrate for kinases.

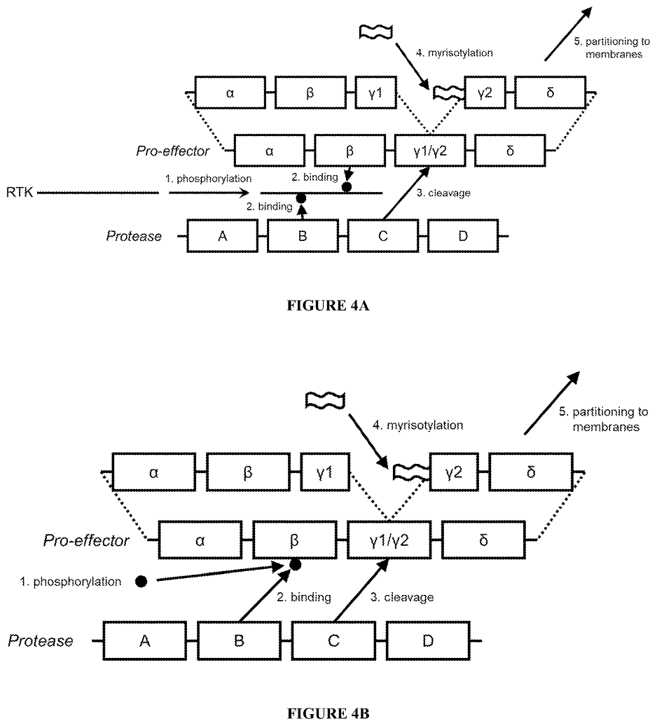

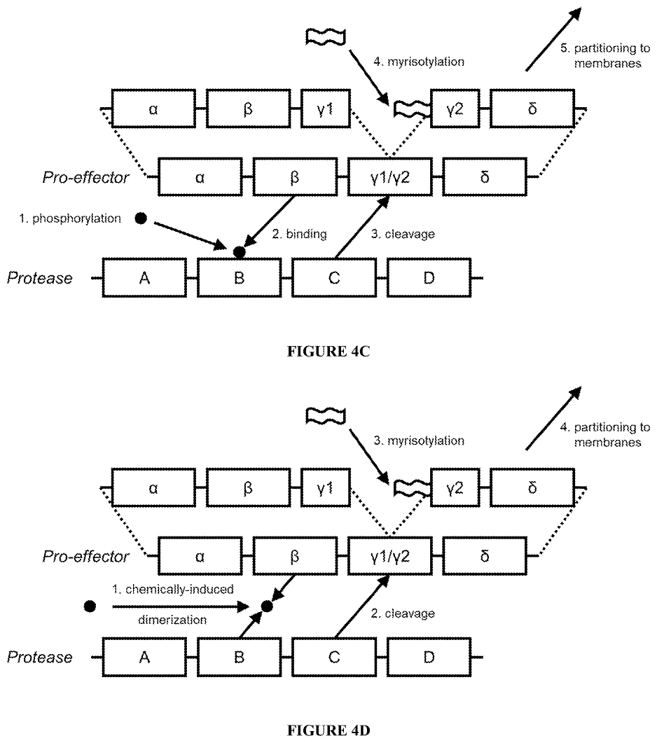

FIGS. 4A-4D provide various examples of the effector/protease combination and mechanism of actions of the system described herein.

For example, and as illustrated in FIG. 4A, when the two proteins of interest (pro-effector and protease), respond to activated tropomyosin-related kinase A (TrkA) through Src-homology domains or phosphotyrosine binding domains within their compositions, it leads to the cleavage of the pro-effector by protease and to the activation of the effector domain, which exerts therapeutic efficacy by modulating desired signaling pathways in the cell.

When the effector component of pro-effector includes a Ras-binding domain or its mutants derived from c-Raf, it responds to activated TrkA and exerts feedback regulation of mitogen-activated protein kinase pathway via sequestration of active Ras. When the effector component of the pro-effector includes a Gq alpha subunit inhibitor derived from phospholipase C beta 3 or mutants, it responds to activated TrkA and exerts negative regulation of Gq signaling pathway via sequestration of active Gq alpha subunit. When the effector component of the pro-effector includes an adenylyl cyclase inhibitor derived from Gi alpha subunits or mutants, responds to activated TrkA and exerts negative regulation of cyclic-AMP signaling pathway via inhibition of adenylyl cyclases. When the effector component of the pro-effector includes a voltage-gated calcium channel inhibitor derived from protein Rem1 or its mutants, responds to activated TrkA and exerts negative regulation of voltage-gated calcium channels via sequestration of their alpha and/or beta subunits. When the effector component of the pro-effector includes other modulators of cellular physiology or signaling pathways, respond to activated TrkA and exert modulation on other cellular physiology or signaling pathways.

As shown in FIGS. 4B and 4C, when the pro-effector and protease include domains sensitive to other receptor tyrosine kinases, they respond to the activated form of other receptor tyrosine kinases and exert modulation on cellular physiology or signaling pathways.

As illustrated in FIG. 4D, when the pro-effector and protease include domains sensitive to other kinases (for example, extracellular signal-regulated kinases, protein kinase A, protein kinase B, protein kinase C, etc.), they respond to the activated form of other kinases and exert modulation on cellular physiology or signaling pathways.

In another embodiment, the invention provides a method of treating chronic pain in a subject including administering to the subject any of the nucleic acid expression vectors described herein, wherein administering includes delivering the expression vector into cells of the subject, thereby treating chronic pain in the subject.

In one aspect, delivering includes using a virus-mediated delivery method or non-viral delivery method.

In some aspects, virus-mediated delivery method includes lentivirus, adeno-associated virus, or herpes simplex virus.

In other aspects, non-viral delivery method includes synthetic mRNA loaded into lipid nanoparticles, synthetic mRNA loaded into exosomes, purified proteins mixed with cell-penetrating peptide or materials, Cas9/CRISPR mediated genome editing, homologous recombination in stem cells, cell transfection, or electroporation.

In one aspect, the chronic pain is induced by osteoarthritis and/or tissue inflammation.

In an additional embodiment, the invention provides a method of treating a subject comprising administering to the subject any of the nucleic acid expression vectors described herein, thereby treating the subject.

In one aspect, the subject has chronic pain, diabetes, an inflammatory disorder, cancer, or disease caused by hyperactive cell signaling pathway.

In yet another embodiment, the invention provides a method of modulating a receptor tyrosine kinase signaling in a cell including contacting the cell with any of the nucleic acid expression vectors described herein, thereby modulating the RTK signaling in the cell.

In one aspect, the RTK is selected from the group consisting of TrkA TrkB, TrkC, ret-c containing neurotrophin receptors, epidermal growth factor receptor (EGFR), insulin receptor, insulin-like growth factor receptor, vascular endothelial growth factor receptor, platelet-derived growth factor, fibroblast growth factor, T-cell receptor and B-cell receptor.

Presented below are examples discussing systems for RTK signaling modulation and uses thereof contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Membrane Anchoring (IMA) System

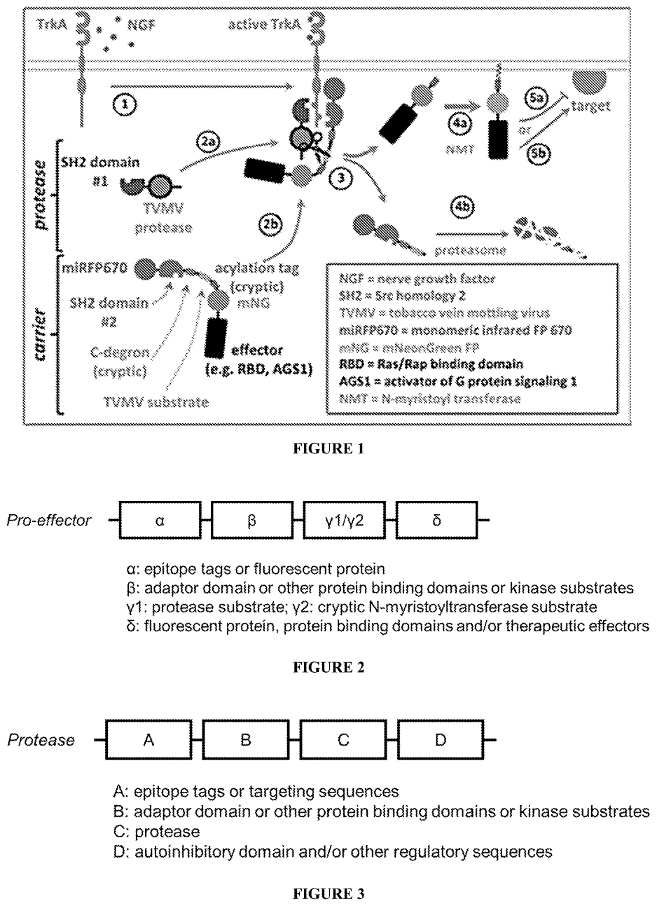

In the iteration of the design, shown in FIG. 1, TrkA was used as the RTK. Recruitment of the protease and the carrier to the activated TrkA are achieved through the inclusion of carefully selected src homology 2 (SH2) domains on each, which bind to phosphorylated tyrosine residues on activated TrkA. The protease is a modified form of the Tobacco Vein Mottling Virus (TVMV) protease. In addition, this system was piloted using two effectors: 1) A modified version of the Ras binding domain (RBD) of the c-Raf protein. This effector should bind and thereby inhibit the activities of Ras and Rap, two pro-hyperalgesic GTPases. 2) Activator of G Protein Signaling 1 (AGS1/DexRas), a protein that activates the anti-hyperalgesic GTPase Gi alpha subunit.

It was demonstrated in heterologously transfected HEK293 cells that TrkA-dependent, protease-dependent, SH2 domain-dependent translocation of a cargo on the carrier to the plasma membrane can be achieved. Using activation of Mitogen-associated Protein Kinase (MAPK) as a readout of Ras-dependent signaling, it was confirmed that acylated RBD inhibits MAPK activation more efficiently than no-acylated RBD. Finally, data showing that when an appropriately tuned RBD is incorporated into the carrier, the full IMA system can be used to achieve TrkA dependent inhibition of MAPK were obtained. AGS1 can also be substituted as the effector to achieve protease-dependent inhibition of forskolin-stimulated adenylate cyclase activity (a target of Gi). this system is being optimized and will be moved it into cultured mouse sensory neurons for proof-of-concept experiments.

First, this system will be optimized in HEK293 cells, then move to cultured mouse dorsal root ganglion neurons, to determine if the cAMP and Ras/Rap pathways in those cells can be controllably modulated in this way. These studies will serve as proof-of-concept for future application of our hyperalgesic signal-shunting system in animal models in vivo and may lead to the development of novel therapies for pain. Furthermore, the modularity of the IMA system makes it adaptable to a wide range of input signals and output effectors. The tools and concepts that were develop may therefore have utility elsewhere in the nervous system and in other settings such as cancer and immunology.

While the system has been used on targeting pain by engaging signaling downstream of TrkA, the system could readily be modified to 1) be driven by RTKs other than TrkA; 2) be driven by more general pro-hyperalgesic signaling events downstream of RTKs and other receptors, through alternative means of promoting proximity between protease and carrier; 3) be dependent on the coincidence of multiple signaling events (e.g., RTK signaling AND intracellular Ca2+ elevations). Preliminary data illustrating the potential feasibility of these expanded applications have been obtained. effectors that target alternative GTPases (e.g., Gq) or that target proteins other than GTPases, could also be substituted as long as engagement of those targets requires membrane localization of the effectors. One example of such a target is the N-type voltage-gated calcium channel, CaV 2.2. Furthermore, pain is far from the only condition in which RTK signaling is pathological. Thus, membrane re-localization of customizable effectors in such an “on-demand” manner that is tuned to prevailing strength of RTK signaling might be useful for the treatment of any number of pathological conditions involving the nervous system, immune system, or cancer cells, among others.

### Example 2

### Inhibition of Signal Transduction Events Promoting Chronic Pain

If delivered into peripheral sensory neurons in vivo, the IMA system could be used to inhibit signal transduction events that would otherwise promote chronic pain.

Examples of pain conditions in which elevations in TrkA signaling are known to occur include osteoarthritis and tissue inflammation. The incorporation of one or more of the three effectors listed above (RBD, AGS1, Gq inhibitor) will likely reduce signaling by Ras/Rap, cAMP-dependent protein kinase, cAMP-activated A Kinase Anchoring Protein (AKAP), and/or Gq-dependent phospholipase C, respectively. The consequence of this is alleviation of pro-hyperalgesic signaling and therefore a reduction in pain.

The components of the IMA system can be delivered either as the functional recruitable protease and pro-effector proteins or as DNA or RNA encoding these proteins

Potential means of delivering the protein or nucleic acid components of the IMA system to sensory neurons include: use of sensory neuron-tropic adeno-associated virus, adenovirus, herpesvirus, or lentiviral vectors; use of DNA plasmids in conjunction with lipid transfection reagents or electroporation; use of nanoparticles or engineered extracellular vesicles; direct injection of components into sensory ganglia or nerves with or without methods such as ultrasound to enhance tissue penetration.

### Example 3

### Tool to Redirect the Signal Transduction Events Downstream of TRKA

The IMA system can be used as an experimental tool to redirect the signal transduction events downstream of TrkA in sensory or sympathetic neurons, in order to better understand how different signaling pathways interact and how signaling homeostasis is achieved, and to determine the cellular consequences of increasing or decreasing the relative strengths among signaling pathways in a coupled fashion. These studies can be performed either in vitro (in neurons derived from laboratory animals, in primary human sensory neurons, or in human iPSC-derived neurons) or in vivo in animal models

The IMA system can be used as a live cell reporter of TrkA activity. In this application, the effector domain consists of a fluorescent protein or a binding partner for a fluorescent small molecule. This provides a means of monitoring TrkA signaling dynamics during pathological processes in complex tissues in real time.

### Example 4

### Alternative Effector Outputs

The IMA system can be adapted to use alternative effector outputs. The key requirement is that the effector's net activity is enhanced by plasma/endosomal membrane targeting. One example of such an effector is a regulator of voltage-gated sodium or calcium channels. This effector provides an alternative means of attenuating pathological pain. Another example is a regulator of the actin cytoskeleton. Use of this effector allows to either attenuate or promote neurite outgrowth and thus facilitate neurological recovery or suppress regeneration-dependent chronic pain.

The inputs necessary for engagement of the IMA system can be made intersectional, to achieve coincidence dependent activity. One example of such an application would be to modify the recruitable protease to exhibit maximal activity only under conditions where intracellular calcium levels are also elevated. In the setting of chronic pain, this ensures that only in neurons with ongoing elevated activity would the IMA system be engaged. This conditional restriction of activity represents a means of diminishing potentially unwanted side effects.

### Example 5

### Tool to Redirect the Signal Transduction

The IMA system could be adapted to respond to input from different receptor tyrosine kinases. By altering the SH2 domains used in the recruitable protease and pro-effector modules, inputs driving the IMA system could be expanded to include other receptor tyrosine kinases, such as: TrkB, TrkC, ret-c containing neurotrophin receptors, epidermal growth factor receptors (EGFR, Her2), insulin receptor, insulin-like growth factor receptor, vascular endothelial growth factor receptor, platelet-derived growth factor, fibroblast growth factor, T-cell and B-cell receptor complexes, to redirect signaling outputs from these receptors. Potential applications of these modified IMA systems might include:
Attenuation of chronic pain mediated by non-TrkA RTKs
Attenuation of glucagon/cAMP dependent hepatic gluconeogenesis in the presence of hyperinsulinemia, in type II diabetes
Suppression of neoplasia in tumor cells exhibiting RTK hyperactivity
Suppression of neovascularization in diabetic retinopathy or cancer
Modulation of brain neuron hyperactivity in epilepsy
Enhancement or reduction in immune cell function in autoimmune diseases, immunodeficiency, inflammatory conditions, infectious diseases, or cancer.

The IMA system could be adapted to respond to non-RTK signaling inputs. Rather than rely on SH2-mediated proximity to promote cleavage of pro-effectors by recruitable protease, one could modify the components of the system to achieve proximity in response to other signaling events such as kinase activity (MAPK, PKC) through phosphorylation-dependent clustering of the recruitable protease and pro-effector. One example application of this approach would be to attenuate pain-promoting signaling pathways in response to elevated MAPK or PKC signaling events.

### Example 6

### Material and Methods

### Cells and Cell Culture

HEK293 cells were cultured in high glucose Dulbecco's Modified Eagles Medium (DMEM) supplemented with 10% fetal bovine serum and 1% penicillin-streptomycin. Cells were plated on poly-L-lysine-coated multiwell glass chamber slides and cultured at 5% CO2/37° C. Transfections were carried out using Lipofectamine 3000 and assays performed one day later. In membrane translocation experiments and for ERK-KTR experiments, stimuli were added directly to culture medium. For calcium imaging experiments, cells were incubated and stimulated with the indicated agents in Calcium Imaging Buffer (CIB) containing (in mM) 130 NaCl, 3 KCL, 2.5 CaCl₂)-2H₂O, 0.6 MgCl₂-6H₂O, 10 HEPES, 1.2 NaHCO₃ and 10 Glucose, adjusted to pH 7.45 with NaOH and to 290-300 mOsm with mannitol. Primary sensory neurons were isolated from adult C57BL6 mice. Briefly, following CO₂ asphyxiation and decapitation, dorsal root ganglia from all levels were surgically isolated, proteolytically digested, mechanically triturated, plated on poly-L-lysine/laminin-coated glass coverslips in medium containing Neurobasal supplemented with B27, and cultured at 5% CO₂/37° C. At the time of plating, cells were incubated with lentiviral particles containing the desired expression constructs.

### Plasmid Constructs

Starting materials for synthesis of carrier, protease, and ERK-KTR cDNAs were obtained as human codon-optimized gene blocks and subcloned into mammalian expression vectors containing the cytomegalovirus promoter. The following cDNAs were obtained from OriGene and subcloned into appropriate expression vectors: mTrkA (MR226042); hTrkA (RC213091), hRET (RC202552), hGFRα1 (RC219943), hEGFR (RG217384), and hErbB2 (RC212583). The following cDNAs were obtained from Addgene: hIGF1R (98344), hPDGFRP (136455), hAxl (105933), mTrkB (83952), mTrkC (24089). Lentiviral constructs were packaged into lentiviral particles in HEK293 cells using helper plasmids psPAX2 (addgene.org/12260/), and VSV-G (addgene.org/8454/). Supernatant was collected after 3 days, particles enriched by ultracentrifugation, and stored at −80C until use.

TABLE 1
Plasmids and Inserts

| | | | SEQ |
|---|---|---|---|
| Plasmid | Key insert | Sequence of key insert | ID NO: |
| JM1242 pC2a- | plasmid with no insert | No insert | |
| puro.dna | | | |
| JM1260_pC2a- | rat TrkA (modified C | MGGRRVRWEVYISRARLVNRQNSAHMSGEAWQ | 1 |
| TrkA-puro.dna | terminus) - P2A - | LQLGAHRRLPRLSEAGAAAMLRGGRRGQLGWHS | |
| | puromycin resistance | WAAGPGSLLAWLILASAGAAPCPDACCPHGSSGL | |
| | | RCTRDGALDSLHHLPGAENLTELYIENQQHLQHL | |
| | | ELRDLRGLGELRNLTIVKSGLRFVAPDAFHFTPRL | |
| | | SRLNLSFNALESLSWKTVQGLSLQELVLSGNPLHC | |
| | | SCALRWLQRWEEEGLGGVPEQKLQCHGQGPLAH | |
| | | MPNASCGVPTLKVQVPNASVDVGDDVLLRCQVE | |
| | | GRGLEQAGWILTELEQSATVMKSGGLPSLGLTLA | |
| | | NVTSDLNRKNLTCWAENDVGRAEVSVQVNVSFP | |
| | | ASVQLHTAVEMHHWCIPFSVDGQPAPSLRWLFN | |
| | | GSVLNETSFIFTEFLEPAANETVRHGCLRLNQPTH | |
| | | VNNGNYTLLAANPFGQASASIMAAFMDNPFEFNP | |
| | | EDPIPDTNSTSGDPVEKKDETPFGVSVAVGLAVFA | |
| | | CLFLSTLLLVLNKCGRRNKFGINRPAVLAPEDGLA | |
| | | MSLHFMTLGGSSLSPTEGKGSGLQGHIIENPQYFS | |
| | | DACVHHIKRRDIVLKWELGEGAFGKVFLAECHNL | |
| | | LPEQDKMLVAVKALKEASESARQDFQREAELLT | |
| | | MLQHQHIVRFFGVCTEGRPLLMVFEYMRHGDLN | |
| | | RFLRSHGPDAKLLAGGEDVAPGPLGLGQLLAVAS | |
| | | QVAAGMVYLAGLHFVHRDLATRNCLVGQGLVV | |
| | | KIGDFGMSRDIYSTDYYRVGGRTMLPIRWMPPESI | |
| | | LYRKFTTESDVWSFGVVLWEIFTYGKQPWYQLSN | |
| | | TEAIDCITQGRELERPRACPPEVYAIMRGCWQREP | |
| | | QQRHSIKDVHARLQALAQAPPVYLDVLGGSGAT | |
| | | NFSLLKQAGDVEENPGPQLTEYKPTVRLATRDDV | |
| | | PRAVRTLAAAFADYPATRHTVDPDRHIERVTELQ | |
| | | ELFLTRVGLDIGKVWVADDGAAVAVWTTPESVE | |
| | | AGAVFAEIGPRMAELSGSRLAAQQQMEGLLAPHR | |
| | | PKEPAWFLATVGVSPDHQGKGLGSAVVLPGVEA | |
| | | AERAGVPAFLETSAPRNLPFYERLGFTVTADVEVP | |
| | | EGPRTWCMTRKPGA* | |
| S1066_pC | mTrkB | MSPWLKWHGPAMARLWGLCLLVLGFWRASLAC | 2 |
| mTrkB.dna | | PTSCKCSSARIWCTEPSPGIVAFPRLEPNSVDPENIT | |
| | | EILIANQKRLEIINEDDVEAYVGLRNLTIVDSGLKF | |
| | | VAYKAFLKNSNLRHINFTRNKLTSLSRRHFRHLDL | |
| | | SDLILTGNPFTCSCDIMWLKTLQETKSSPDTQDLY | |
| | | CLNESSKNMPLANLQIPNCGLPSARLAAPNLTVEE | |
| | | GKSVTLSCSVGGDPLPTLYWDVGNLVSKHMNET | |
| | | SHTQGSLRITNISSDDSGKQISCVAENLVGEDQDS | |
| | | VNLTVHFAPTITFLESPTSDHHWCIPFTVRGNPKP | |
| | | ALQWFYNGAILNESKYICTKIHVTNHTEYHGCLQ | |
| | | LDNPTHMNNGDYTLMAKNEYGKDERQISAHFMG | |
| | | RPGVDYETNPNYPEVLYEDWTTPTDIGDTTNKSN | |
| | | EIPSTDVADQSNREHLSVYAVVVIASVVGFCLLV | |
| | | MLLLLKLARHSKFGMKGPASVISNDDDSASPLHHI | |
| | | SNGSNTPSSSEGGPDAVIIGMTKIPVIENPQYFGI | |
| | | TNSQLKPDTFVQHIKRHNIVLKRELGEGAFGKVFL | |
| | | AECYNLCPEQDKILVAVKTLKDASDNARKDFHRE | |
| | | AELLTNLQHEHIVKFYGVCVEGDPLIMVFEYMKH | |
| | | GDLNKFLRAHGPDAVLMAEGNPPTELTQSQMLHI | |
| | | AQQIAAGMVYLASQHFVHRDLATRNCLVGENLL | |
| | | VKIGDFGMSRDVYSTDYYRVGGHTMLPIRWMPP | |
| | | ESIMYRKFTTESDVWSLGVVLWEIFTYGKQPWYQ | |
| | | LSNNEVIECITQGRVLQRPRTCPQEVYELMLGCW | |
| | | QREPHTRKNIKSIHTLLQNLAKASPVYLDILGADP | |
| | | K* | |
| S1067_pLV-C | PYR(Mandi)-Ca | MVRSLYKSAGGPMPSELTPEERSELKNSIAEFHTY | 3 |
| PYR(Mandi)- | sensitive TVMV | QLDPGSCSSLHAQRIHAPPELVWSIVRRFDKPQTH | |
| CaTVMVp2.dna | protease - P2A | RHFIKSCSVEQNFEMRVGCTRDIIVISGLPANTST | |
| | | ERLDILDDERRVTGASIIGGEHRLTNYKGVTTVHR | |
| | | FEKENRIWTVVLESYVVDMPEGNSEDDTRMLADT | |
| | | VVKLNLQKLATVAEAMARNSGDGSGSQVTFGGG | |
| | | MHGGSAGGSAGGSAGGSSSGSKALLKGVRDFNPI | |
| | | SACVCLLENSSDGHSERLFGIGFGPYIIANQHLFRR | |
| | | NNGELTIKTMHLPDQLTEEQIAEYKEAFSLFDKDG | |
| | | DGTITTKELGTVMRSLGHNPTEAELQDMINEVDA | |
| | | DGDGTIDFPEFLTMMARKMKYRGGVKLIPSWTT | |
| | | VILVKSMLRKRSFGNPFGGDTEEEIREAFGVFDKD | |
| | | GNGYISAAELRHVMTNLGEKLTDEEVDEMIREAD | |
| | | IDGDGQVNYEEFVQMMTAKAAAGEFKVKNSTQL | |
| | | QMKPVEGRDIIVIKMAKDFPPFPQKLKFRQPTIKD | |
| | | RVCMVSTNFQQKSVSSLVSESSHIVHKEDTSFWQ | |
| | | HWITTKDGQCGSPLVSIIDGNILGIHSLTHTTNGSN | |
| | | YFVEFPEKFVATYLDAADGWCKNWKFNADKISW | |
| | | GSFILWEDAPEDFMGSGASGSGATNFSLLKQAGD | |
| | | VEENPGPGTAIAGFKHASRVTN* | |
| S1093_pLV- | miR670FP-ABI-degron | MGLFGAPVAGHASGSPAFGTASHSNCEHEEIHLA | 4 |
| CAG 670-ABI- | -TVMVcleavage site | GSIQPHGALLVVSEHDHRVIQASANAAEFLNLGS | |
| Cde-TVMVs(G)- | (G) - acyl tag - mNG | VLGVPLAEIDGDLLIKILPHLDPTAEGMPVAVRCR | |
| acyl-mNG.dna | | IGNPSTEYCGLMHRPPEGGLIIELERAGPSIDLSGT | |
| | | LAPALERIRTAGSLRALCDDTVLLFQQCTGYDRV | |
| | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYKGIDGVPLYGFTS | |
| | | ICGRRPEMEAAVSTIPRFLQSSSGSMLDGRFDPQS | |
| | | AAHFFGVYDGHGGSQVANYCRERMHLALAEEIA | |
| | | KEKPMLCDGDTWLEKWKKALFNSFLRVDSEIESV | |
| | | APETVGSTSVVAVVFPSHIFVANCGDSRAVLCRG | |
| | | KTALPLSVDHKPDREDEAARIEAAGGKVIQWNGA | |
| | | RVFGVLAMSRSIGDRYLKPSIIPDPEVTAVKRVKE | |
| | | DDCLILASDGVWDVMTDEEACEMARKRILLWHK | |
| | | KNAVAGDASLLADERRKEGKDPAAMSAAEYLSK | |
| | | LAIQRGSKDNISVVVVDLKGSAGSGFEGGELYPY | |
| | | DVPDYAGGLPMSCAQESITSLYKKAGSETVRFQG | |
| | | CINSKRRDTKLTEEREQKLISEEDLGAPVSKGEED | |
| | | NMASLPATHELHIFGSINGVDFDMVGQGTGNPND | |
| | | GYEELNLKSTKGDLQFSPWILVPHIGYGFHQYLPY | |
| | | PDGMSPFQAAMVDGSGYQVHRTMQFEDGASLTV | |
| | | NYRYTYEGSHIKGEAQVKGTGFPADGPVMTNSLT | |
| | | AADWCRSKKTYPNDKTIISTFKWSYTTGNGKRYR | |
| | | STARTTYTFAKPMAANYLKNQPMYVFRKTELKH | |
| | | SKTELNFKEWQKAFTDVMGMDELYKGIEEFGLE* | |
| S1094_pLV- | miR670FP-ABI- | MGLFGAPVAGHASGSPAFGTASHSNCEHEEIHLA | 5 |
| CAG 670-ABI- | degron-TVMV | GSIQPHGALLVVSEHDHRVIQASANAAEFLNLGS | |
| Cde-TVMVs(G)- | cleavage site (G)-acyl | VLGVPLAEIDGDLLIKILPHLDPTAEGMPVAVRCR | |
| acyl-mNG- | tag-mNG-GqI(21,A)- | IGNPSTEYCGLMHRPPEGGLIIELERAGPSIDLSGT | |
| GqI(21,A).dna | flexible linker | LAPALERIRTAGSLRALCDDTVLLFQQCTGYDRV | |
| | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYKGIDGVPLYGFTS | |
| | | ICGRRPEMEAAVSTIPRFLQSSSGSMLDGRFDPQS | |
| | | AAHFFGVYDGHGGSQVANYCRERMHLALAEEIA | |
| | | KEKPMLCDGDTWLEKWKKALFNSFLRVDSEIESV | |
| | | APETVGSTSVVAVVFPSHIFVANCGDSRAVLCRG | |
| | | KTALPLSVDHKPDREDEAARIEAAGGKVIQWNGA | |
| | | RVFGVLAMSRSIGDRYLKPSIIPDPEVTAVKRVKE | |
| | | DDCLILASDGVWDVMTDEEACEMARKRILLWHK | |
| | | KNAVAGDASLLADERRKEGKDPAAMSAAEYLSK | |
| | | LAIQRGSKDNISVVVVDLKGSAGSGFEGGELYPY | |
| | | DVPDYAGGLPMSCAQESITSLYKKAGSETVRFQG | |
| | | CINSKRRDTKLTEEREQKLISEEDLGAPVSKGEED | |
| | | NMASLPATHELHIFGSINGVDFDMVGQGTGNPND | |
| | | GYEELNLKSTKGDLQFSPWILVPHIGYGFHQYLPY | |
| | | PDGMSPFQAAMVDGSGYQVHRTMQFEDGASLTV | |
| | | NYRYTYEGSHIKGEAQVKGTGFPADGPVMTNSLT | |
| | | AADWCRSKKTYPNDKTIISTFKWSYTTGNGKRYR | |
| | | STARTTYTFAKPMAANYLKNQPMYVFRKTELKH | |
| | | SKTELNFKEWQKAFTDVMGMDELYKGIDGHQDY | |
| | | AEALANPIKHVSLMDQRGSAGSAAGFEEFGLE* | |
| S462_pC 670- | miR670FP-degron- | MGLFGAPVAGHASGSPAFGTASHSNCEHEEIHLA | 6 |
| Cde- | TVMVcleavage site | GSIQPHGALLVVSEHDHRVIQASANAAEFLNLGS | |
| TVMVs(M)- | (M) - acyl tag - mNG - | VLGVPLAEIDGDLLIKILPHLDPTAEGMPVAVRCR | |
| acyl-mNG- | FKBP | IGNPSTEYCGLMHRPPEGGLIIELERAGPSIDLSGT | |
| FKBP ires | | LAPALERIRTAGSLRALCDDTVLLFQQCTGYDRV | |
| hyg.dna | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYGGGQLYPYDVPD | |
| | | YAGGLPMSCAQESITSLYKKAGSETVRFQMGCIN | |
| | | SKRRDTKLTEEREQKLISEEDLGAPVSKGEEDNM | |
| | | ASLPATHELHIFGSINGVDFDMVGQGTGNPNDGY | |
| | | EELNLKSTKGDLQFSPWILVPHIGYGFHQYLPYPD | |
| | | GMSPFQAAMVDGSGYQVHRTMQFEDGASLTVN | |
| | | YRYTYEGSHIKGEAQVKGTGFPADGPVMTNSLTA | |
| | | ADWCRSKKTYPNDKTIISTFKWSYTTGNGKRYRS | |
| | | TARTTYTFAKPMAANYLKNQPMYVFRKTELKHS | |
| | | KTELNFKEWQKAFTDVMGMDELYKSAGGGVQV | |
| | | ETISPGDGRTFPKRGQTCVVHYTGMLEDGKKFDS | |
| | | SRDRNKPFKFMLGKQEVIRGWEEGVAQMSVGQR | |
| | | AKLTISPDYAYGATGHPGIIPPHATLVFDVELLKLE | |
| | | SAGGSAGGSAGGSAGGSAGEFGLE* | |
| S463_pC 670- | miR670FP-degron- | MGLFGAPVAGHASGSPAFGTASHSNCEHEEIHLA | 7 |
| Cde-TVMVs(G)- | TVMV cleavage | GSIQPHGALLVVSEHDHRVIQASANAAEFLNLGS | |
| acyl-mNG- | site(G)-acyl tag-mNG- | VLGVPLAEIDGDLLIKILPHLDPTAEGMPVAVRCR | |
| FKBP ires | FKBP | IGNPSTEYCGLMHRPPEGGLIIELERAGPSIDLSGT | |
| hyg.dna | | LAPALERIRTAGSLRALCDDTVLLFQQCTGYDRV | |
| | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYGGGQLYPYDVPD | |
| | | YAGGLPMSCAQESITSLYKKAGSETVRFQGCINSK | |
| | | RRDTKLTEEREQKLISEEDLGAPVSKGEEDNMAS | |
| | | LPATHELHIFGSINGVDFDMVGQGTGNPNDGYEE | |
| | | LNLKSTKGDLQFSPWILVPHIGYGFHQYLPYPDG | |
| | | MSPFQAAMVDGSGYQVHRTMQFEDGASLTVNY | |
| | | RYTYEGSHIKGEAQVKGTGFPADGPVMTNSLTAA | |
| | | DWCRSKKTYPNDKTIISTFKWSYTTGNGKRYRST | |
| | | ARTTYTFAKPMAANYLKNQPMYVFRKTELKHSK | |
| | | TELNFKEWQKAFTDVMGMDELYKSAGGGVQVE | |
| | | TISPGDGRTFPKRGQTCVVHYTGMLEDGKKFDSS | |
| | | RDRNKPFKFMLGKQEVIRGWEEGVAQMSVGQRA | |
| | | KLTISPDYAYGATGHPGIIPPHATLVFDVELLKLES | |
| | | AGGSAGGSAGGSAGGSAGEFGLE* | |
| S485 pC 670- | mi670FP-degron- | MGLFGAPVAGHASGSPAFGTASHSNCEHEEIHLA | 8 |
| Cde- | TVMV cleavage site | GSIQPHGALLVVSEHDHRVIQASANAAEFLNLGS | |
| TVMVs(M)- | (M)-acyl tag-mNG | VLGVPLAEIDGDLLIKILPHLDPTAEGMPVAVRCR | |
| acyl-mNG.dna | | IGNPSTEYCGLMHRPPEGGLIIELERAGPSIDLSGT | |
| | | LAPALERIRTAGSLRALCDDTVLLFQQCTGYDRV | |
| | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYGGGQLYPYDVPD | |
| | | YAGGLPMSCAQESITSLYKKAGSETVRFQMGCIN | |
| | | SKRRDTKLTEEREQKLISEEDLGAPVSKGEEDNM | |
| | | ASLPATHELHIFGSINGVDFDMVGQGTGNPNDGY | |
| | | EELNLKSTKGDLQFSPWILVPHIGYGFHQYLPYPD | |
| | | GMSPFQAAMVDGSGYQVHRTMQFEDGASLTVN | |
| | | YRYTYEGSHIKGEAQVKGTGFPADGPVMTNSLTA | |
| | | ADWCRSKKTYPNDKTIISTFKWSYTTGNGKRYRS | |
| | | TARTTYTFAKPMAANYLKNQPMYVFRKTELKHS | |
| | | KTELNFKEWQKAFTDVMGMDELYKGIEEFGLE* | |
| S492_pC 670- | miR670FP-SH2(from | MGLFGAPVAGHASGSPAFGTASHSNCEHEEIHLA | 9 |
| SH2(SH2B1)- | SH2B1)-degron-TVMV | GSIQPHGALLVVSEHDHRVIQASANAAEFLNLGS | |
| Cde- | cleavage site (M)-acyl | VLGVPLAEIDGDLLIKILPHLDPTAEGMPVAVRCR | |
| TVMVs(M)- | tag-mNG | IGNPSTEYCGLMHRPPEGGLIIELERAGPSIDLSGT | |
| acyl-mNG.dna | | LAPALERIRTAGSLRALCDDTVLLFQQCTGYDRV | |
| | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYKGIDGMGGEGDQ | |
| | | PLSGYPWFHGMLSRLKAAQLVLTGGTGSHGVFL | |
| | | VRQSETRRGEYVLTFNFQGKAKHLRLSLNEEGQC | |
| | | RVQHLWFQSIFDMLEHFRVHPIPLESGGSSDVVLV | |
| | | SYVPSQRQQGREQAGSGSAGSAAGSGFEELYPYD | |
| | | VPDYAGGLPMSCAQESITSLYKKAGSETVRFQMG | |
| | | CINSKRRDTKLTEEREQKLISEEDLGAPVSKGEED | |
| | | NMASLPATHELHIFGSINGVDFDMVGQGTGNPND | |
| | | GYEELNLKSTKGDLQFSPWILVPHIGYGFHQYLPY | |
| | | PDGMSPFQAAMVDGSGYQVHRTMQFEDGASLTV | |
| | | NYRYTYEGSHIKGEAQVKGTGFPADGPVMTNSLT | |
| | | AADWCRSKKTYPNDKTIISTFKWSYTTGNGKRYR | |
| | | STARTTYTFAKPMAANYLKNQPMYVFRKTELKH | |
| | | SKTELNFKEWQKAFTDVMGMDELYKGIEEFGLE* | |
| S576_pC | 3XNES-HA- | MVDIKEKLCYVALDSLEEELDVLVLDDKVAEKLE | 10 |
| 3×NES-670-Cde- | miR670FP-degron- | ALSVRGYPYDVPDYAGRVTMGLFGAPVAGHASG | |
| TVMVs(M)- | TVMVcleavage site | SPAFGTASHSNCEHEEIHLAGSIQPHGALLVVSEH | |
| acyl-mNG- | (M)-acyl tag-mNG- | DHRVIQASANAAEFLNLGSVLGVPLAEIDGDLLIK | |
| RBD(CRaf,T68 | RBD(CRaf,T68A) | ILPHLDPTAEGMPVAVRCRIGNPSTEYCGLMHRPP | |
| A) ires hyg.dna | | EGGLIIELERAGPSIDLSGTLAPALERIRTAGSLRAL | |
| | | CDDTVLLFQQCTGYDRVMVYRFDEQGHGLVFSE | |
| | | CHVPGLESYFGNRYPSSTVPQMARQLYVRQRVR | |
| | | VLVDVTYQPVPLEPRLSPLTGRDLDMSGCFLRSM | |
| | | SPCHLQFLKDMGVRATLAVSLVVGGKLWGLVVC | |
| | | HHYLPRFIRFELRAICKRLAERIATRITALESGMDE | |
| | | LYKAYGGGQLYPYDVPDYAGGLPMSCAQESITSL | |
| | | YKKAGSETVRFQMGCINSKRRDTKLTEEREQKLIS | |
| | | EEDLGAPVSKGEEDNMASLPATHELHIFGSINGVD | |
| | | FDMVGQGTGNPNDGYEELNLKSTKGDLQFSPWIL | |
| | | VPHIGYGFHQYLPYPDGMSPFQAAMVDGSGYQV | |
| | | HRTMQFEDGASLTVNYRYTYEGSHIKGEAQVKG | |
| | | TGFPADGPVMTNSLTAADWCRSKKTYPNDKTIIS | |
| | | TFKWSYTTGNGKRYRSTARTTYTFAKPMAANYL | |
| | | KNQPMYVFRKTELKHSKTELNFKEWQKAFTDVM | |
| | | GMDELYKGIDNTIRVFLPNKQRAVVNVRNGMSL | |
| | | HDCLMKALKVRGLQPECCAVFRLLHEHKGKKAR | |
| | | LDWNTDAASLIGEELQVDFLGSAGSAAGSGFEEF | |
| | | GGALE* | |
| S579_pC | 3XNES-HA- | MVDIKEKLCYVALDSLEEELDVLVLDDKVAEKLE | 11 |
| 3×NES-670- | miR670FP-SH2(from | ALSVRGYPYDVPDYAGRVTMGLFGAPVAGHASG | |
| SH2(SH2B1)- | SH2B1)-degron-TVMV | SPAFGTASHSNCEHEEIHLAGSIQPHGALLVVSEH | |
| Cde- | cleavage site (M)-acyl | DHRVIQASANAAEFLNLGSVLGVPLAEIDGDLLIK | |
| TVMVs(M)- | tag-mNG-RBD(from C- | ILPHLDPTAEGMPVAVRCRIGNPSTEYCGLMHRPP | |
| acyl-mNG- | Raf,mutant T68A) | EGGLIIELERAGPSIDLSGTLAPALERIRTAGSLRAL | |
| RBD(CRaf,T68 | | CDDTVLLFQQCTGYDRVMVYRFDEQGHGLVFSE | |
| A).dna | | CHVPGLESYFGNRYPSSTVPQMARQLYVRQRVR | |
| | | VLVDVTYQPVPLEPRLSPLTGRDLDMSGCFLRSM | |
| | | SPCHLQFLKDMGVRATLAVSLVVGGKLWGLVVC | |
| | | HHYLPRFIRFELRAICKRLAERIATRITALESGMDE | |
| | | LYKAYKGIDGMGGEGDQPLSGYPWFHGMLSRLK | |
| | | AAQLVLTGGTGSHGVFLVRQSETRRGEYVLTFNF | |
| | | QGKAKHLRLSLNEEGQCRVQHLWFQSIFDMLEHF | |
| | | RVHPIPLESGGSSDVVLVSYVPSQRQQGREQAGS | |
| | | GSAGSAAGSGFEELYPYDVPDYAGGLPMSCAQES | |
| | | ITSLYKKAGSETVRFQMGCINSKRRDTKLTEEREQ | |
| | | KLISEEDLGAPVSKGEEDNMASLPATHELHIFGSIN | |
| | | GVDFDMVGQGTGNPNDGYEELNLKSTKGDLQFS | |
| | | PWILVPHIGYGFHQYLPYPDGMSPFQAAMVDGSG | |
| | | YQVHRTMQFEDGASLTVNYRYTYEGSHIKGEAQ | |
| | | VKGTGFPADGPVMTNSLTAADWCRSKKTYPNDK | |
| | | TIISTFKWSYTTGNGKRYRSTARTTYTFAKPMAA | |
| | | NYLKNQPMYVFRKTELKHSKTELNFKEWQKAFT | |
| | | DVMGMDELYKGIDNTIRVFLPNKQRAVVNVRNG | |
| | | MSLHDCLMKALKVRGLQPECCAVFRLLHEHKGK | |
| | | KARLDWNTDAASLIGEELQVDFLGSAGSAAGSGF | |
| | | EEFGGALE* | |
| S622_pC | 3XNES-GA24- | MVDIKEKLCYVALDSLEEELDVLVLDDKVAEKLE | 12 |
| 3xNES-GA24- | Bipartite SH2 (from | ALSVRGGGAGAGGAGGAGGAGAGGAGAGARSL | |
| 2xSH2(PLCg1)- | PLCg1)-TVMV | YKGIDGHSNEKWFHGKLGAGRDGRHIAERLLTEY | |
| TVMV-AI ires | protease- | CIETGAPDGSFLVRESETFVGDYTLSFWRNGKVQ | |
| hyg.dna | Autoinhibitory | HCRIHSRQDAGTPKFFLTDNLVFDSLYDLITHYQQ | |
| | domain -P2A | VPLRCNEFEMRLSEPVPQTNAHESKEWYHASLTR | |
| | | AQAEHMLMRVPRDGAFLVRKRNEPKSYAISFRAE | |
| | | GKIKHCRVQQEGQTVMLGNSEFDSLVDLISYYEK | |
| | | HPLYRKMKLRYPINEEGSAGSAAGFEEFGSAGSA | |
| | | AGSGMHGGSAGGSAGGSAGGSSSGSKALLKGVR | |
| | | DFNPISACVCLLENSSDGHSERLFGIGFGPYIIANQ | |
| | | HLFRRNNGELTIKTMHGEFKVKNSTQLQMKPVEG | |
| | | RDIIVIKMAKDFPPFPQKLKFRQPTIKDRVCMVST | |
| | | NFQQKSVSSLVSESSHIVHKEDTSFWQHWITTKD | |
| | | GQCGSPLVSIIDGNILGIHSLTHTTNGSNYFVEFPE | |
| | | KFVATYLDAADGWCKNWKFNADKISWGSFILWE | |
| | | DAPEDFMSGLVPKGVGREYVRFAPGSGASGSGAT | |
| | | NFSLLKQAGDVEENPGPGTAIAGFKHASRVGR* | |
| S715_pC 670- | miR670FP-degron - | MGLFGAPVAGHASGSPAFGTASHSNCEHEEIHLA | 13 |
| Cde- | TVMVcleavage site | GSIQPHGALLVVSEHDHRVIQASANAAEFLNLGS | |
| TVMVs(M)- | (M) - acyl tag - mNG - | VLGVPLAEIDGDLLIKILPHLDPTAEGMPVAVRCR | |
| acyl-mNG- | GID1-flexible linker | IGNPSTEYCGLMHRPPEGGLIIELERAGPSIDLSGT | |
| GID1.dna | | LAPALERIRTAGSLRALCDDTVLLFQQCTGYDRV | |
| | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYGGGQLYPYDVPD | |
| | | YAGGLPMSCAQESITSLYKKAGSETVRFQMGCIN | |
| | | SKRRDTKLTEEREQKLISEEDLGAPVSKGEEDNM | |
| | | ASLPATHELHIFGSINGVDFDMVGQGTGNPNDGY | |
| | | EELNLKSTKGDLQFSPWILVPHIGYGFHQYLPYPD | |
| | | GMSPFQAAMVDGSGYQVHRTMQFEDGASLTVN | |
| | | YRYTYEGSHIKGEAQVKGTGFPADGPVMTNSLTA | |
| | | ADWCRSKKTYPNDKTIISTFKWSYTTGNGKRYRS | |
| | | TARTTYTFAKPMAANYLKNQPMYVFRKTELKHS | |
| | | KTELNFKEWQKAFTDVMGMDELYKGIDGMMAA | |
| | | SDEVNLIESRTVVPLNTWVLISNFKVAYNILRRPD | |
| | | GTFNRHLAEYLDRKVTANANPVDGVFSFDVLIDR | |
| | | RINLLSRVYRPAYADQEQPPSILDLEKPVDGDIVP | |
| | | VILFFHGGSFAHSSANSAIYDTLCRRLVGLCKCVV | |
| | | VSVNYRRAPENPYPCAYDDGWIALNWVNSRSWL | |
| | | KSKKDSKVHIFLAGDSSGGNIAHNVALRAGESGID | |
| | | VLGNILLNPMFGGNERTESEKSLDGKYFVTVRDR | |
| | | DWYWKAFLPEGEDREHPACNPFSPRGKSLEGVSF | |
| | | PKSLVVVAGLDLIRDWQLAYAEGLKKAGQEVKL | |
| | | MHLEKATVGFYLLPNNNHFHNVMDEISAFVNAE | |
| | | CGSAGSAAGSGFEEFGLE* | |
| S722_pC GAI- | GAI-TVMV protease- | MKRDHHHHHHQDKKTMMMNEEDDGNGMDELL | 14 |
| TVMVp-AI.dna | Autoinhibitory domain- | AVLGYKVRSSEMADVAQKLEQLEVMMSNVQED | |
| | P2A site | DLSQLATETVHYNPAELYTWLDSMLTDLNFGGG | |
| | | MHGGSAGGSAGGSAGGSSSGSKALLKGVRDFNPI | |
| | | SACVCLLENSSDGHSERLFGIGFGPYIIANQHLFRR | |
| | | NNGELTIKTMHGEFKVKNSTQLQMKPVEGRDIIVI | |
| | | KMAKDFPPFPQKLKFRQPTIKDRVCMVSTNFQQK | |
| | | SVSSLVSESSHIVHKEDTSFWQHWITTKDGQCGSP | |
| | | LVSIIDGNILGIHSLTHTTNGSNYFVEFPEKFVATY | |
| | | LDAADGWCKNWKFNADKISWGSFILWEDAPEDF | |
| | | MSGLVPKGVGREYVRFAPGSGASGSGATNFSLLK | |
| | | QAGDVEENPGPGTAIAGFKHASRVGR* | |
| S754_pCMV6 | mTrkA(K547A) - myc - | MGGRRVRWEVYISRARLVNRQNFVIRLTIGRPGIR | 15 |
| mTrkA(K547A)- | flag | RLDPVPRRSAAAIAMLRGQRLGQLGWHRPAAGL | |
| mycDDK.dna | | GSLMTSLMLACASAASCREVCCPVGPSGLRCTRA | |
| | | GSLDTLRGLRGAGNLTELYVENQQHLQRLEFEDL | |
| | | QGLGELRSLTIVKSGLRFVAPDAFRFTPRLSHLNL | |
| | | SSNALESLSWKTVQGLSLQDLTLSGNPLHCSCALF | |
| | | WLQRWEQEGLCGVHTQTLHDSGPGDQFLPLGHN | |
| | | TSCGVPTVKIQMPNDSVEVGDDVFLQCQVEGLAL | |
| | | QQADWILTELEGAATVKKFGDLPSLGLILVNVTS | |
| | | DLNKKNVTCWAENDVGRAEVSVQVSVSFPASVH | |
| | | LGLAVEQHHWCIPFSVDGQPAPSLRWLFNGSVLN | |
| | | ETSFIFTQFLESALTNETMRHGCLRLNQPTHVNNG | |
| | | NYTLLAANPYGQAAASVMAAFMDNPFEFNPEDPI | |
| | | PVSFSPVDGNSTSRDPVEKKDETPFGVSVAVGLA | |
| | | VSAALFLSALLLVLNKCGQRSKFGINRPAVLAPED | |
| | | GLAMSLHFMTLGGSSLSPTEGKGSGLQGHIMENP | |
| | | QYFSDTCVHHIKRQDIILKWELGEGAFGKVFLAEC | |
| | | YNLLNDQDKMLVAVAALKEASENARQDFQREAE | |
| | | LLTMLQHQHIVRFFGVCTEGGPLLMVFEYMRHG | |
| | | DLNRFLRSHGPDAKLLAGGEDVAPGPLGLGQLLA | |
| | | VASQVAAGMVYLASLHFVHRDLATRNCLVGQGL | |
| | | VVKIGDFGMSRDIYSTDYYRVGGRTMLPIRWMPP | |
| | | ESILYRKFSTESDVWSFGVVLWEIFTYGKQPWYQ | |
| | | LSNTEAIECITQGRELERPRACPPDVYAIMRGCWQ | |
| | | REPQQRLSMKDVHARLQALAQAPPSYLDVLGTR | |
| | | TRPLEQKLISEEDLAANDILDYKDDDDKV* | |
| S758_pC | 3XNES-GA24- | MVDIKEKLCYVALDSLEEELDVLVLDDKVAEKLE | 16 |
| 3xNES-GA24- | Bipartite SH2(from | ALSVRGGGAGAGGAGGAGGAGAGGAGAGARSL | |
| 2xSH2(PLCg1)- | PLCg1)-TVMV | YKGIDGHSNEKWFHGKLGAGRDGRHIAERLLTEY | |
| TVMVpx(inactiv | protease (inactive)- | CIETGAPDGSFLVRESETFVGDYTLSFWRNGKVQ | |
| e)-AI ires | Autoinhibitory domain- | HCRIHSRQDAGTPKFFLTDNLVFDSLYDLITHYQQ | |
| hyg.dna | P2A | VPLRCNEFEMRLSEPVPQTNAHESKEWYHASLTR | |
| | | AQAEHMLMRVPRDGAFLVRKRNEPKSYAISFRAE | |
| | | GKIKHCRVQQEGQTVMLGNSEFDSLVDLISYYEK | |
| | | HPLYRKMKLRYPINEEGSAGSAAGFEEFGSAGSA | |
| | | AGSGMHGGSAGGSAGGSAGGSSSGSKALLKGVR | |
| | | DFNPISACVCLLENSSDGHSERLFGIGFGPYIIANQ | |
| | | HLFRRNNGELTIKTMHGEFAVANSTQLQMKPVEG | |
| | | RDIIVIKMAKDFPPFPQKLKFRQPTIKDRVCMVST | |
| | | NFQQKSVSSLVSESSHIVHKEDTSFWQHWITTKD | |
| | | GQAGSPLVSIIDGNILGIHSLTHTTNGSNYFVEFPE | |
| | | KFVATYLDAADGWCKNWKFNADKISWGSFILWE | |
| | | DAPEDFMSGLVPKGVGREYVRFAPGSGASGSGAT | |
| | | NFSLLKQAGDVEENPGPGTAIAGFKHASRVGR* | |
| S843_pLV-CAG | GAI-TVMV protease- | MKRDHHHHHHQDKKTMMMNEEDDGNGMDELL | |
| GAI-TVMVp- | Autoinhibitory domain | AVLGYKVRSSEMADVAQKLEQLEVMMSNVQED | |
| AI.dna | - P2A domain | DLSQLATETVHYNPAELYTWLDSMLTDLNFGGG | |
| | | MHGGSAGGSAGGSAGGSSSGSKALLKGVRDFNPI | |
| | | SACVCLLENSSDGHSERLFGIGFGPYIIANQHLFRR | |
| | | NNGELTIKTMHGEFKVKNSTQLQMKPVEGRDIIVI | |
| | | KMAKDFPPFPQKLKFRQPTIKDRVCMVSTNFQQK | |
| | | SVSSLVSESSHIVHKEDTSFWQHWITTKDGQCGSP | |
| | | LVSIIDGNILGIHSLTHTTNGSNYFVEFPEKFVATY | |
| | | LDAADGWCKNWKFNADKISWGSFILWEDAPEDF | |
| | | MSGLVPKGVGREYVRFAPGSGASGSGATNFSLLK | |
| | | QAGDVEENPGPGTAIAGFKHASRVTN* | |
| S844 pLV-CAG | miR670FP-degron - | MGLFGAPVAGHASGSPAFGTASHSNCEHEEIHLA | 17 |
| 670-Cde- | TVMVcleavage site | GSIQPHGALLVVSEHDHRVIQASANAAEFLNLGS | |
| TVMVs(M)- | (M) - acyl tag - mNG - | VLGVPLAEIDGDLLIKILPHLDPTAEGMPVAVRCR | |
| acyl-mNG- | GID1-flexible linker | IGNPSTEYCGLMHRPPEGGLIIELERAGPSIDLSGT | |
| GID1.dna | | LAPALERIRTAGSLRALCDDTVLLFQQCTGYDRV | |
| | | MVYRFDEQGHGLVFSECHVPGLESYFGNRYPSST | |
| | | VPQMARQLYVRQRVRVLVDVTYQPVPLEPRLSPL | |
| | | TGRDLDMSGCFLRSMSPCHLQFLKDMGVRATLA | |
| | | VSLVVGGKLWGLVVCHHYLPRFIRFELRAICKRL | |
| | | AERIATRITALESGMDELYKAYGGGQLYPYDVPD | |
| | | YAGGLPMSCAQESITSLYKKAGSETVRFQMGCIN | |
| | | SKRRDTKLTEEREQKLISEEDLGAPVSKGEEDNM | |
| | | ASLPATHELHIFGSINGVDFDMVGQGTGNPNDGY | |
| | | EELNLKSTKGDLQFSPWILVPHIGYGFHQYLPYPD | |
| | | GMSPFQAAMVDGSGYQVHRTMQFEDGASLTVN | |
| | | YRYTYEGSHIKGEAQVKGTGFPADGPVMTNSLTA | |
| | | ADWCRSKKTYPNDKTIISTFKWSYTTGNGKRYRS | |
| | | TARTTYTFAKPMAANYLKNQPMYVFRKTELKHS | |
| | | KTELNFKEWQKAFTDVMGMDELYKGIDGMMAA | |
| | | SDEVNLIESRTVVPLNTWVLISNFKVAYNILRRPD | |
| | | GTFNRHLAEYLDRKVTANANPVDGVFSFDVLIDR | |
| | | RINLLSRVYRPAYADQEQPPSILDLEKPVDGDIVP | |
| | | VILFFHGGSFAHSSANSAIYDTLCRRLVGLCKCVV | |
| | | VSVNYRRAPENPYPCAYDDGWIALNWVNSRSWL | |
| | | KSKKDSKVHIFLAGDSSGGNIAHNVALRAGESGID | |
| | | VLGNILLNPMFGGNERTESEKSLDGKYFVTVRDR | |
| | | DWYWKAFLPEGEDREHPACNPFSPRGKSLEGVSF | |
| | | PKSLVVVAGLDLIRDWQLAYAEGLKKAGQEVKL | |
| | | MHLEKATVGFYLLPNNNHFHNVMDEISAFVNAE | |
| | | CGSAGSAAGSGFEEFGLE* | |
| S717_pC | FRBstb-CaTVMV-PA2 | MWHEGLEEASRLYFGERNVKGMFEVLEPLHAM | 18 |
| FRBstb- | | MERGPQTLKETSFNQAYGRDLMEAQEWCRKYM | |
| CaTVMVp2 ires | | KSGNVKDLTQAWDLYYHVFRRISKQFGGGMHGG | |
| hyg.dna | | SAGGSAGGSAGGSSSGSKALLKGVRDFNPISACV | |
| | | CLLENSSDGHSERLFGIGFGPYIIANQHLFRRNNGE | |
| | | LTIKTMHLPDQLTEEQIAEYKEAFSLFDKDGDGTI | |
| | | TTKELGTVMRSLGHNPTEAELQDMINEVDADGD | |
| | | GTIDFPEFLTMMARKMKYRGGVKLIPSWTTVILV | |
| | | KSMLRKRSFGNPFGGDTEEEIREAFGVFDKDGNG | |
| | | YISAAELRHVMTNLGEKLTDEEVDEMIREADIDG | |
| | | DGQVNYEEFVQMMTAKAAAGEFKVKNSTQLQM | |
| | | KPVEGRDIIVIKMAKDFPPFPQKLKFRQPTIKDRVC | |
| | | MVSTNFQQKSVSSLVSESSHIVHKEDTSFWQHWI | |
| | | TTKDGQCGSPLVSIIDGNILGIHSLTHTTNGSNYFV | |
| | | EFPEKFVATYLDAADGWCKNWKFNADKISWGSF | |
| | | ILWEDAPEDFMGSGASGSGATNFSLLKQAGDVEE | |
| | | NPGPGTAIAGFKHASRVGR* | |
| S917_pLV-C | mTrkB | MDVSLCPAKCSFWRIFLLGSVWLDYVGSVLACPA | 19 |
| mTrkC.dna | | NCVCSKTEINCRRPDDGNLFPLLEGQDSGNSNGN | |
| | | ASINITDISRNITSIHIENWRGLHTLNAVDMELYTG | |
| | | LQKLTIKNSGLRNIQPRAFAKNPHLRYINLSSNRLT | |
| | | TLSWQLFQTLSLRELRLEQNFFNCSCDIRWMQLW | |
| | | QEQGEARLDSQSLYCISADGSQLPLFRMNISQCDL | |
| | | PEISVSHVNLTVREGDNAVITCNGSGSPLPDVDWI | |
| | | VTGLQSINTHQTNLNWTNVHAINLTLVNVTSEDN | |
| | | GFTLTCIAENVVGMSNASVALTVYYPPRVVSLVE | |
| | | PEVRLEHCIEFVVRGNPTPTLHWLYNGQPLRESKII | |
| | | HMDYYQEGEVSEGCLLFNKPTHYNNGNYTLIAK | |
| | | NALGTANQTINGHFLKEPFPESTDFFDFESDASPTP | |
| | | PITVTHKPEEDTFGVSIAVGLAAFACVLLVVLFIMI | |
| | | NKYGRRSKFGMKGPVAVISGEEDSASPLHHINHGI | |
| | | TTPSSLDAGPDTVVIGMTRIPVIENPQYFRQGHNC | |
| | | HKPDTYVQHIKRRDIVLKRELGEGAFGKVFLAEC | |
| | | YNLSPTKDKMLVAVKALKDPTLAARKDFQREAE | |
| | | LLTNLQHEHIVKFYGVCGDGDPLIMVFEYMKHG | |
| | | DLNKFLRAHGPDAMILVDGQPRQAKGELGLSQM | |
| | | LHIASQIASGMVYLASQHFVHRDLATRNCLVGAN | |
| | | LLVKIGDFGMSRDVYSTDYYRVGGHTMLPIRWM | |
| | | PPESIMYRKFTTESDVWSFGVILWEIFTYGKQPWF | |
| | | QLSNTEVIECITQGRVLERPRVCPKEVYDVMLGC | |
| | | WQREPQQRLNIKEIYKILHALGKATPIYLDILG* | |

### Chemicals

Chemical stimulus agents used in these experiments and their final working concentrations were as follows:
NGF (recombinant murine β-NGF, Pepro Tech 450-34) 100 nM
Rapalog AP21967 (Takara 635056) 500 nM in 0.1% DMSO
GA3-AM (Tocris 5407) 100 uM in 0.1% DMSO
Mandipropamid (Sigma Aldrich, 5 uM in 0.1% DMSO)
A23187 (Sigma Aldrich, 1 uM in 0.01% DMSO)
ATP (Sigma Aldrich, 200 uM)
Carbachol (Sigma Aldrich, 100 uM)
Thapsigargin (Sigma Aldrich T9033, 1 uM)
K Gluconate (100 mM)

### mNG Membrane Translocation and ERK-KTR Imaging

Imaging was performed using an EVOS M7000 microscope equipped with an automated stage and both a 40× lens and a 20× lens. Imaging with excitation and emission wavelengths appropriate for each fluorophore was carried out at ambient temperature (−25° C.).

### Calcium Imaging

Cells were loaded with Fura2-AM (1 uM) dissolved in CIB and imaged on a Nikon TE200 inverted microscope equipped with a cMOS camera (NEO, Andor) and an excitation filter wheel (Mac5000, Ludl) as described (Ostrow et al., Scientific Reports (2019) 9:13098). Stimuli were delivered by gravity-driven perfusion using an electronically controlled valve manifold.

### Image Analysis

Fiji (Image J) and NIS Elements (Nikon) software programs were used for image analysis. To quantify membrane localization, mNG mean fluorescence intensity was measured within a roughly toroidal area closely encompassing the plasma membrane (PM) and within a parallel area within the cell cortex of the cytoplasm (C). The PM/C ratio was calculated and in some cases, was normalized to the C/N ratio of a co-expressed acyl-tagged mCherry fluorophore. For ERK-KTR, the cytoplasmic/nuclear ratio (C/N) was calculated from mean intensities within cytoplasmic and nuclear regions of interest in a given cell. These were defined by co-expressing a fluorescent fluorophore that localized either to the nucleus or to the endoplasmic reticulum, with the latter defining the nucleus by exclusion. Relative calcium concentrations were inferred from the ratio of 520 nm Fura2 fluorescence emission upon 340 excitation/emission upon 380 nm excitation. Data from imaging software were exported to Excel (Microsoft) and then to Prism (Graphpad) to permit statistical analysis and graphing.

### Example 7

### TRKA-IMA-Mediated Map Kinase Signaling Inhibition

To demonstrate the principle of the IMA system, HEK293 were transfected cells with cDNA vectors encoding the following components: 1) human TrkA; 2) Protease construct (Tobacco Vein Mottling Virus protease containing an autoinhibitory domain to attenuate its activity and fused to a Src-homology 2 (SH2) domain derived from phospholipase C gamma 1 (PLCγ1 SH2NC); and 3) Proeffector construct containing, in sequence, fluorescent protein miRFP670-SH2 domain from SH2B1—TVMV substrate domain—cryptic acylation tag—fluorescent protein mNeonGreen (mNG).

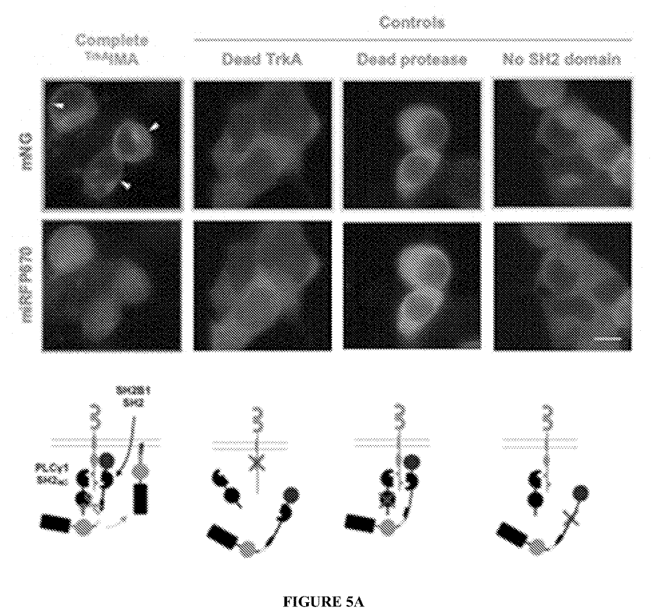

As illustrated in FIG. 5A, when the complete TrkAIMA system was introduced, the constitutive activity of TrkA resulted in proteolytic cleavage of the Proeffector, with resultant acylation of the fluorescent mNG component and translocation of that protein to the inner leaflet of the plasma membrane, in addition to other undefined membrane loci. However, utilization of a kinase-dead TrkA mutant (TrkA K547A), utilization of a catalytically inactive and stabilized TVMV protease (K65A, K67A, C151A) (ncbi.nlm.nih.gov/pmc/articles/PMC3005794/), or omission of the SH2 domain from the Proeffector resulted in the mNG remained homogeneously distributed in the cytoplasm. The constitutive activity of TrkA resulted in MNG translocation to the plasma membrane (PM) when all components were intact. Less plasma membrane localization was observed if TrkA lacked kinase activity, or the TVMV protease was catalytically inactive or if the proeffector lacked an SH2 adaptor domain (see also FIG. 5B for quantification). These results demonstrate the dependence of proeffector translocation on all IMA components. Data shown reflect Mean±SEM normalized plasma membrane enrichment derived from 4-5 independent wells. One-way ANOVA. FIGS. 5A-5B demonstrate TrkA-IMA-mediated translocation of proeffector to the plasma membrane. Together, these findings illustrate the necessity of the multiple components of the IMA system for receptor-driven membrane translocation of the Proeffector.

Next, to determine whether the IMA system could be utilized to inhibit a cellular signaling target, a modified Ras binding domain (RBD^{T68A}) from c-Raf was incorporated into the Proeffector. This variant exhibits a 10× reduction in affinity towards active Ras. A fluorescent kinase translocation reporter (KTR) was also co-expressed to provide a downstream readout of Ras activity. Following Ras-dependent activation of the mitogen-activated protein kinase ERK 1/2, the subcellular distribution of the ERK-KTR reporter shifts from a predominantly nuclear localization to a predominantly cytoplasmic localization. Thus, nuclear exclusion of the reporter reflects high levels of ERK activity.

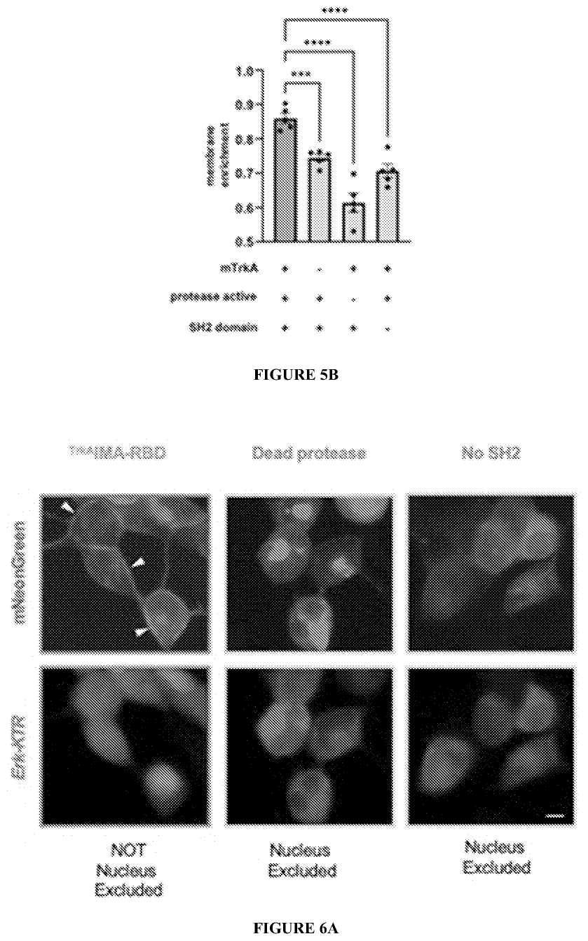

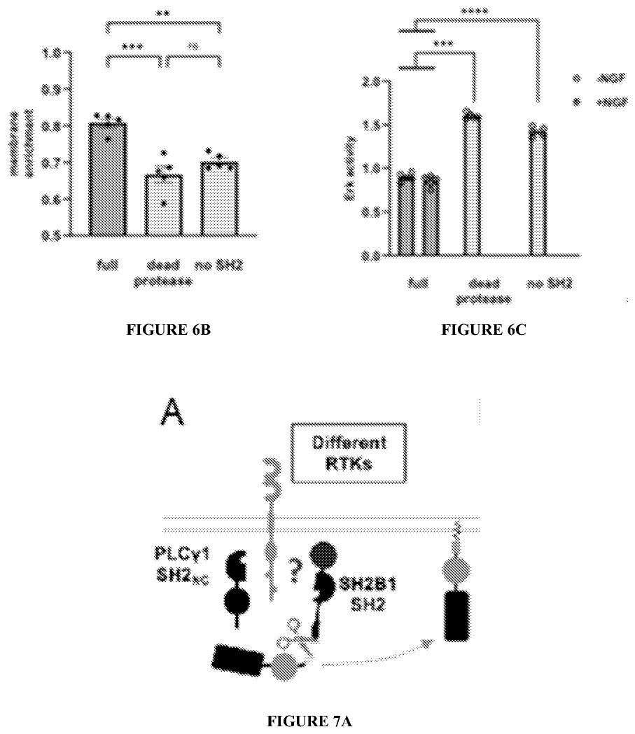

HEK293 cells were then transfected with mouse TrkA, a protease construct, a proeffector construct containing a modified ras binding domain (RBD) from c-Raf, and a Kinase Translocation Reporter that shows an increase in cytoplasmic/nuclear (C/N) ratio when the MAP kinase ERK is activated. As shown in FIGS. 6A-6C, the presence of intact versions of all ^{TrkA}IMA components resulted in plasma membrane enrichment of the mNG signal and an EKR-KTR cytoplasmic/nuclear (C/N) ratio <1. In contrast, when a catalytically inactive protease was used or if the SH2 domain was omitted from the Proeffector, mNG failed to become enriched at the cell surface and the ERK-KTR exhibited a significantly higher C/N ratio.

In this experiment, it was shown that constitutive TrkA activity performs two functions: 1) stimulates ras-dependent ERK activation; and 2) drives the IMA reaction to simultaneously activate PM translocation of the RBD-containing pro-effector and thus simultaneous Ras inhibition. Note that inclusion of all IMA components results in surface translocation of mNG-labeled proeffector and a C/N ratio close to 1, whereas inclusion of an inactive TVMV protease or omission of the SH2 adaptor from the proeffector results in relatively higher C/N ratio, reflecting a less inhibited TrkA-triggered Ras activation. Data shown reflect Mean±SEM normalized plasma membrane enrichment derived from 4 independent wells. One-way ANOVA.

These data are consistent with inhibition of Ras-driven ERK activation by the intact IMA components. Inclusion of the TrkA ligand, nerve growth factor (100 g/ml, 15 min) failed to further reduce the C/N ratio, suggesting that the constitutive activity of the overexpressed TrkA was sufficient to maximally suppress Ras and thus ERK activity.

FIGS. 6A-6C demonstrate of TrkAIMA-mediated inhibition of Ras-dependent MAP Kinase signaling.

### Example 8

IMA with Alternative RTKs Targets

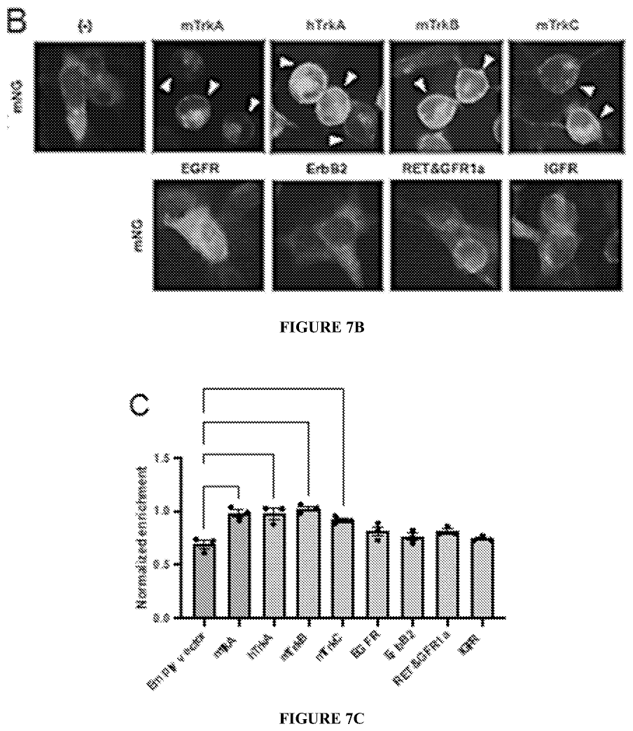

Because the binding affinities of SH2 domains for different receptor tyrosine kinases (RTKs) vary, the relative abilities of several RTKs to drive IMA-mediated Proeffector membrane translocation were next sought to be determined. To do so, the RTKs shown in FIG. 7A were overexpressed, along with the Protease and Proeffector components. The IMA components used including SH2 adaptor domains are indicated in FIG. 7A.

Examination mNG translocation revealed that, whereas mouse and human TrkA, mouse TrkB, and mouse TrkC could all induce an increase in mNG enrichment at the plasma membrane, relative to a condition lacking any transfected RTK, no significant membrane enrichment was observed in the presence of any of the other RTKs tested.

FIG. 7B shows the representative images obtained with co-expression of the indicated RTKs, a normalized plasma membrane mNG enrichment being quantified for each (FIG. 7C). Data points represent individual coverslips. Mean±SEM, one-way ANOVA.

Together, these findings demonstrate that IMA-mediated Proeffector translocation, in the setting of the two specific SH2 domains used here, differs based upon the specific RTK that is overexpressed.

Next whether IMA-mediated Proeffector cleavage and translocation using alternative inputs could be achieved was determined. One approach employed was to use small molecule chemical dimerizers to bring the Protease and Proeffector into close proximity. As a proof of concept for this chemical dimerization induced IMA (^{CD}IMA) approach, two different dimerizable systems were tested: 1) the FRB-FKBP system, in which these two protein domains can be approximated using either of two small molecules, rapamycin or rapalog; and 2) the GID1-GAI system, in which these two protein domains can be approximated using the plant hormone derivative gibberellic acid methoxy ester (GA3-AM). Following replacement of the SH2 domains in the Protease and Proeffector components with the respective protein domain pairs, these two components were expressed in HEK293 cells and the distribution of the mNG fluorescence was monitored without vs with the addition of rapalog (500 nM) or GA3-AM (100 uM) to the cells.

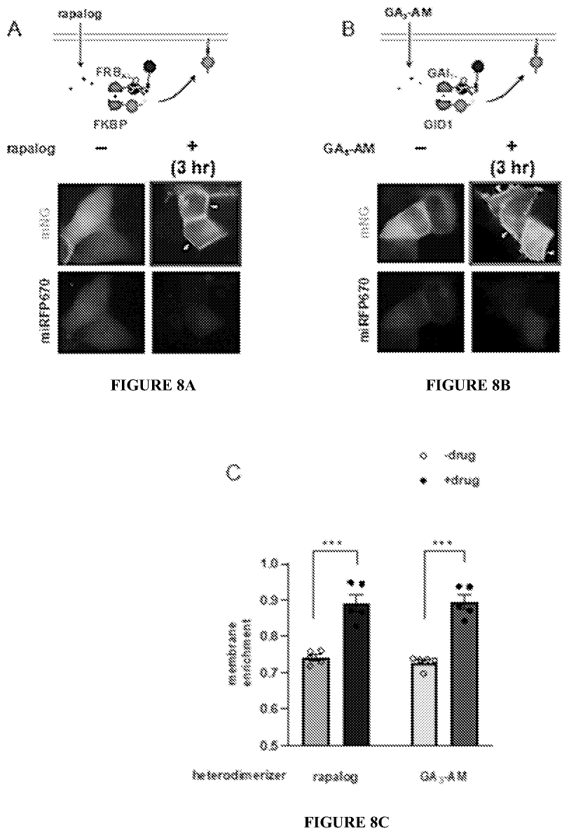

As illustrated in FIGS. 8A-8C, the chemical dimerization induced translocation of mNG to membrane via IMA was evaluated. Rapalog induced dimerization of FRB and FKBP drives translocation (FIG. 8A), and Gibberellic Acid derivative (GA3-AM) induced dimerization of GAI1 and GID1 induces translocation (FIG. 8B). miRFP670 was used as a non-translocated control. Quantification of mNG membrane enrichment where data points represent individual coverslips is shown in FIG. 8C. Mean±SEM, two-tailed t-test. Either small molecule could induce an increase in plasma membrane enrichment of mNG by 3 hrs of treatment. Thus, the IMA system can be driven by small molecule chemical dimerizers.

One potential advantage of IMA is that its engagement can be made dependent on endogenous cellular conditions. While one approach to introducing such a level of control is exemplified by the ^{TrkA}IMA system, described above, an alternative approach (^{cD+ca2+}IMA) was developed that relies on the coincident presence of two signals: 1) an exogenously administered small molecule; and 2) elevated intracellular Ca²⁺ levels. Ca²⁺ elevation was chosen as a trigger for this system because it is a common feature of many pathological processes, including elevated neuronal activity, and thus might constitute a valuable conditional requirement for hypothetical therapeutic applications of the IMA system. To impose this dual requirement for activity, a modification similar to that applied to the Tobacco Etch Virus protease (TEV) by Sanchez et al. (PNAS, 2020, 117 (52): 33186-33196), in which the activity of the protease was made dependent on elevations in free Ca²⁺ levels, was applied to the TVMV protease.

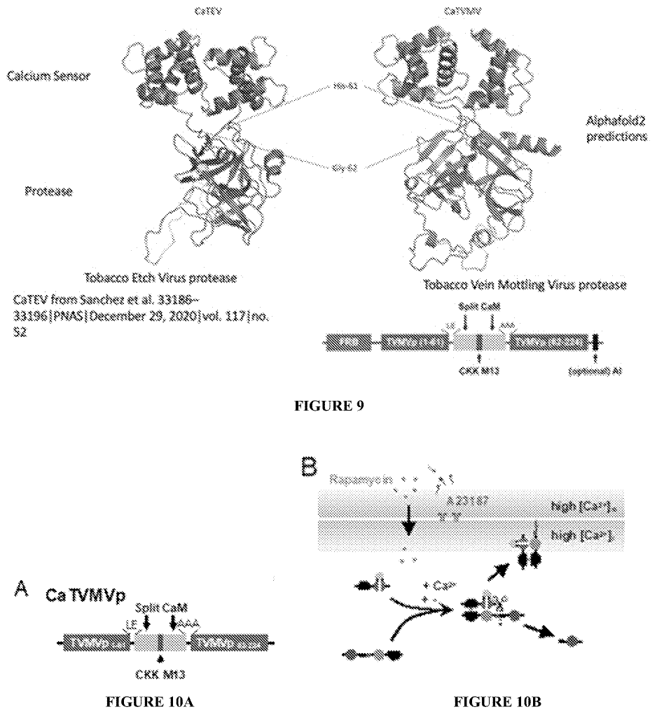

As shown in FIG. 9, this strategy involved splitting the protease coding sequence and interposing a Ca²⁺-sensitive cassette derived from the Ca²⁺ reporter GCAMP7s. In the absence of Ca²⁺ binding, the protease adopts a loose structure that fails to support efficient proteolytic activity. Upon Ca²⁺ binding to the Ca2+ sensor domain, the two portions of the protease are brought together to restore a functional catalytic capability. After using Alphafold2 to model the structures of TEV and TVMV, an analogous modification was applied to the latter protease, inserting the Ca²⁺ sensor cassette into a structurally analogous position of TVMV, and utilizing the Ca²⁺ sensor domain from GCAMP8s instead of GCAMP7s. The resulting protease, CaTVMV, exhibited a partially compromised proteolytic efficiency, assessed using the IMA membrane translocation readout (not shown). To restore an acceptable dynamic range of activity, the autoinhibitory domain that normally dampens protease function was therefore removed.

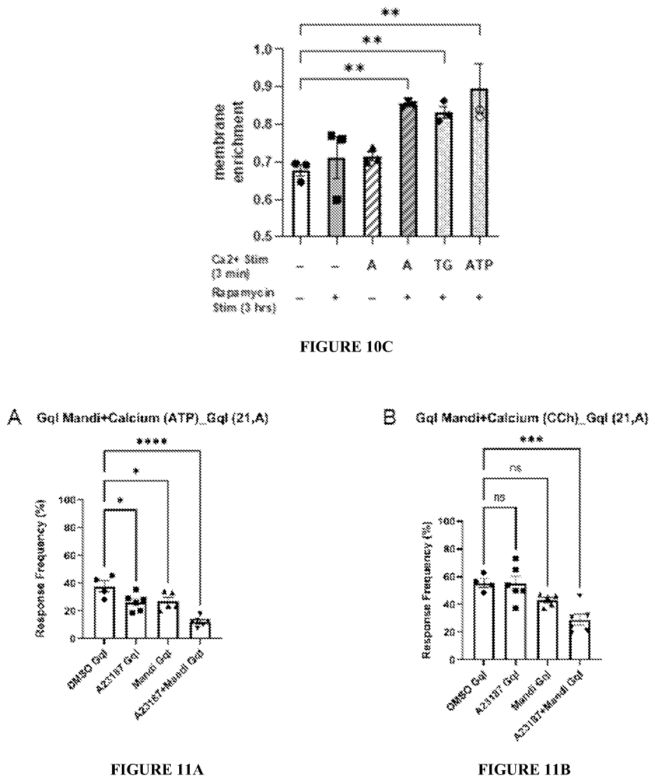

FIGS. 10A-10B show the result of an experiment with a pilot ^{CID+Ca2+}IMA system. In the example shown, rapamycin was used as the chemical dimerizer. HEK293 cells were transfected with a Protease construct consisting of an FRB-CaTVMV fusion and a Proeffector consisting of a fusion of FKBP—TVMV substrate domain—cryptic acylation tag—mNG. As a further modification of this system, a TVMV substrate domain variant (containing glycine at position P1′) that is relatively permissive for protease action was utilized, to further facilitate cleavage by the relatively weak modified protease. Cells were stimulated with or without Rapamycin for 3 hrs, and then stimulated for an additional 3 min with one of three triggers for elevated intracellular [Ca2+]: 1) Ca²⁺ ionophore A23187, to mediate Ca²⁺ influx from the extracellular buffer; 2) Thapsigargin, to block Ca²⁺ pumps in the endoplasmic reticulum and thus passively allow leakage of Ca²⁺ into the cytoplasm; and 3) Adenosine triphosphate (ATP) to stimulate Gq-coupled purinergic receptors and thereby trigger release of Ca²⁺ from intracellular stores through the generation of inositol triphosphate (IP3). Quantification of plasma membrane enrichment of the mNG carried on the Proeffector revealed that the combination of rapamycin with any of the three mediators of Ca²⁺ elevation produced the largest membrane translocation, consistent with a dual requirement of this system for rapamycin and Ca²⁺.

As shown in FIGS. 10A-10B IMA mediated translocation of mNG to plasma membrane was driven by dual treatment with chemical dimerizer and elevated Ca2+.

Based on this positive result, whether ^{CID+Ca2+}IMA could be used to inhibit a targeted signal transduction pathway was investigated. Because rapamycin might not be an ideal chemical dimerizer for use in some cell types, due to its interactions with endogenous FKBP, an alternative chemically induced dimerization system, namely the dimerization of the PYR and ABI protein domains by the small agrochemical mandipropamid (Mandi) was utilized. As a downstream effector, a peptide (GqI I860A) previously shown to inhibit Gq family signaling proteins was used. Like Ras, Gq is a membrane-associated GTPase that is a key contributor to a number of pathophysiological processes, and thus a logical target for the IMA approach. A prior publication had also shown that the GqI peptide we chose to incorporate into our Proeffector was more efficacious as a Gq inhibitor when containing a CAAX prenylation motif, to achieve membrane targeting, making this effector ideal for our system.

FIGS. 11A-11B show the results of an experiment in which HEK293 cells were transfected with two constructs encoding: 1) Protease consisting of PYR-CaTVMV; and 2) Proeffector consisting of miRFP670-ABI-TVMV substrate site—cryptic acylation site—mNG—GqI. The ^{CID+Ca2+}IMA system was activated by the treatment of cells with A23187 and/or Mandi or with vehicle (DMSO) as a negative control. To achieve a physiological readout of Gq activity, cells were loaded with the Ca²⁺ sensing dye Fura2, and then treated with one of two ligands for endogenous Gq-coupled receptors, ATP and Carbachol. It is important to note that in this experiment, since Ca²⁺ serves both as a trigger for ^{CID+Ca2+}IMA-mediated Proeffector cleavage and as the physiological readout of downstream effects, these two events needed to be separated in time. Thus, cells were treated with A23187 and/or Mandi first, then after a sufficient washout period to permit Ca²⁺ levels to return to baseline, the ATP or carbachol challenge was performed.

As shown in FIGS. 11A-11B, the combination of A23187 and Mandi produced the most evident decrease in Ca²⁺ response to either ATP or Carbachol, relative to the vehicle control. This finding confirms that the ^{CID+Ca2+}IMA system provides a feasible strategy for achieving Gq inhibition in a coincident signal-dependent manner.

A “chemical plus Ca2+”-dependent IMA system was used to inhibit Gq signaling. HEK cells were transfected with IMA components containing a Gq inhibitor peptide in the pro-effector. Effector plasma membrane translocation is dependent on mandipropamid induced dimerization of protease and effector, plus Ca2+ elevation to activate protease. After loading with the Ca2+ sensor Fura2, Cells were imaged and stimulated with ATP and carbachol (CCh), two ligands that activate endogenous Gq-coupled receptors, to evoke Ca2+ release via IP3 generation. As illustrated in FIG. 11A and FIG. 11B, percent cells responding to ATP and percent cells responding to carbachol, were evaluated. Data points represent individual coverslips. Mean±SEM, one-way ANOVA.

Next whether IMA could be used to achieve inducible membrane localization of protein domains in primary mouse sensory neurons was determined. Neurons dissociated from adult wild-type mouse dorsal root ganglia were plated onto poly-L-lysine/laminin-coated coverslips and infected with lentiviruses carrying the IMA components, with stimulation experiments performed three days later.

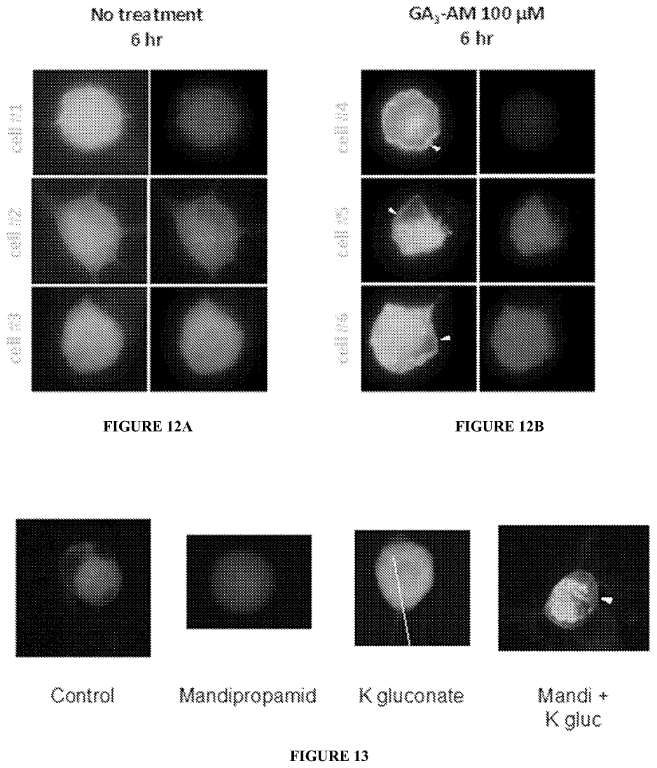

FIGS. 12A and 12B show an experiment in which ^{CD}IMA, was utilized with GA3-AM as the trigger, to achieve translocation of mNG to the plasma membrane. In FIG. 13, the ^{CID+Ca2+}IMA system, with Mandi as the chemical dimerizer and K⁺ gluconate as the inducer of elevated Ca²⁺ levels was utilized. In this latter experiment, both stimuli were applied for three hrs prior to imaging of the cells. Note that only in the setting of both Mandi and K⁺ gluconate does the illustrated cell show membrane translocation of mNG. Thus, IMA mediated effector translocation can be achieved in primary sensory neurons in response to either chemical dimerizer or a combination of dimerizer plus elevated Ca²⁺. These findings lay a solid foundation for the application of IMA to signal transduction modulation in neurons.

As illustrated in FIGS. 12A and 12B, GA3-AM inducible IMA mediated translocation of mNeon Green to the plasma membrane (arrowheads) of cultured mouse DRG neurons. Green channel is mNG. Red channel is control miR670FP.

As shown in FIG. 13, dual stimulus conditional induction of membrane translocation of IMA proeffector mNeon Green in cultured mouse DRG neuron was performed. Neurons were lentivirally transduced with IMA components dependent on both mandipropamid dimerization and elevation of Ca2+(achieved by K gluconate-stimulated membrane depolarization). Example neurons are shown.

Although the invention has been described with reference to the above examples, it will be understood that modifications and variations are encompassed within the spirit and scope of the invention. Accordingly, the invention is limited only by the following claims.

1. An expression vector comprising:
(a) a nucleic acid sequence encoding a pro-effector protein, and
(b) a nucleic acid sequence encoding a protease,
wherein the pro-effector protein is a fusion protein comprising of a spacer protein, a protease substrate, a peptide substrate for post-translational acylation, and an effector protein, and
wherein the protease of interest is a fusion protein containing a protease.

2. (canceled)

3. The expression vector of claim 1, wherein the expression vector comprises pS622, pS458, pS459, pS462, pS463, pS485, pS498, pS492, pS634, pS577, pS579, pS630, pS631.

4. The expression vector of claim 1, wherein the spacer protein is a single- or a multi-component, comprising one or more of (i) a fluorescent protein, (ii) a SNAP Tag, (iii) a HaloTag, (iv) an epitope tag, (v) a dimerization domain for kinase substrates, (vi) a binding domain for kinase substrates, (vii) a peptide substrate for kinases, (viii) a proteolysis-sensitive destabilization domain or (ix) a combination thereof.

5. The expression vector of claim 4, wherein the dimerization domain is selected from the group consisting of a FK506-binding protein, a Gibberellin receptor 1, and an abscisic acid receptor ABI1.

6. The expression vector of claim 4, wherein the binding domain for kinase substrates is selected from the group consisting of a Src-homology 2 domain, a phosphotyrosine binding domain, a Forkhead-associated 1, and a WW domain.

7. The expression vector of claim 4, wherein the proteolysis-sensitive destabilization domain is derived from mouse ornithine decarboxylase.

8. (canceled)

9. The expression vector of claim 1, wherein the peptide substrate for post-translational acylation is a substrate of methionine aminopeptidase and/or N-myristoyltransferase.

10. The expression vector of claim 1, wherein the effector protein comprises one or more components of (i) a Ras-binding domain derived from c-Raf or mutants thereof, (ii) a Gq alpha subunit inhibitor derived from phospholipase C beta 3 or mutants thereof, (iii) an adenylyl cyclase inhibitor derived from Gi alpha subunits or mutants thereof, (iv) a voltage-gated calcium channel inhibitor derived from protein Rem1 or mutants thereof, (v) any other effector domain, (vi) a fluorescent protein, (vii) a SNAP Tag, a HaloTag, (viii) an epitope tag, (ix) a protein domain for chemically induced dimerization or (x) any combination thereof.

11. The expression vector of claim 10, wherein the protein domain for chemically induced dimerization is selected from the group consisting of FK506-binding protein, Gibberellin receptor 1, and abscisic acid receptor ABI1.

12. The expression vector of claim 1, wherein the protease comprises a plant virus-derived protease.

13. The expression vector of claim 12, wherein the plant virus-derived protease is a tobacco etch virus (TEV) protease, tobacco vein mottling virus (TVMV) protease, or any mutant thereof.

14. The expression vector of claim 12, wherein the plant virus-derived protease is a modified form with an attached autoinhibitory domain, or a modified form sensitive to calcium.

15. The expression vector of claim 14, wherein the autoinhibitory domain is EYVRFAP.

16. The expression vector of claim 1, wherein the fusion protein containing a protease further comprises one or more components selected from the group consisting of a protein domain for chemically induced dimerization, a binding domain for kinase substrates, and a peptide substrate for kinases.

17. A method of treating chronic pain in a subject comprising administering to the subject the nucleic acid expression vector of claim 1, wherein administering comprises delivering the expression vector into cells of the subject, thereby treating chronic pain in the subject.

18. The method of claim 17, wherein delivering comprises using a virus-mediated delivery method or non-viral delivery method.

19. (canceled)

20. (canceled)

21. The method of claim 18, wherein the chronic pain is induced by osteoarthritis and/or tissue inflammation.

22. A method of treating chronic pain, diabetes, an inflammatory disorder, cancer, or disease caused by hyperactive cell signaling pathway in a subject comprising administering to the subject the nucleic acid expression vector of claim 1, thereby treating the subject.

23. (canceled)

24. A method of modulating a receptor tyrosine kinase (RTK) signaling in a cell comprising contacting the cell with the nucleic acid expression vector of any of claim 1, thereby modulating the RTK signaling in the cell.

25. The method of claim 24, wherein the RTK is selected from the group consisting of TrkA TrkB, TrkC, ret-c containing neurotrophin receptors, epidermal growth factor receptor (EGFR), insulin receptor, insulin-like growth factor receptor, vascular endothelial growth factor receptor, platelet-derived growth factor, fibroblast growth factor, T-cell receptor and B-cell receptor.


SEQUENCE LISTING (21 sequences)

SEQ ID NO: 1 - protein, 1063 aa, synthetic construct
MGGRRVRWEVYISRARLVNRQNSAHMSGEAWQLQLGAHRRLPRLSEAGAAAMLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNLTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLGGSGATNFSLLKQAGDVEENPGPQLTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 2 - protein, 825 aa, synthetic construct
MSPWLKWHGPAMARLWGLCLLVLGFWRASLACPTSCKCSSARIWCTEPSPGIVAFPRLEPNSVDPENITEILIANQKRLEIINEDDVEAYVGLRNLTIVDSGLKFVAYKAFLKNSNLRHINFTRNKLTSLSRRHFRHLDLSDLILTGNPFTCSCDIMWLKTLQETKSSPDTQDLYCLNESSKNMPLANLQIPNCGLPSARLAAPNLTVEEGKSVTLSCSVGGDPLPTLYWDVGNLVSKHMNETSHTQGSLRITNISSDDSGKQISCVAENLVGEDQDSVNLTVHFAPTITFLESPTSDHHWCIPFTVRGNPKPALQWFYNGAILNESKYICTKIHVTNHTEYHGCLQLDNPTHMNNGDYTLMAKNEYGKDERQISAHFMGRPGVDYETNPNYPEVLYEDWTTPTDIGDTTNKSNEIPSTDVADQSNREHLSVYAVVVIASVVGFCLLVMLLLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHTRKNIKSIHTLLQNLAKASPVYLDILGADPK

SEQ ID NO: 3 - protein, 675 aa, synthetic construct
MVRSLYKSAGGPMPSELTPEERSELKNSIAEFHTYQLDPGSCSSLHAQRIHAPPELVWSIVRRFDKPQTHRHFIKSCSVEQNFEMRVGCTRDIIVISGLPANTSTERLDILDDERRVTGASIIGGEHRLTNYKGVTTVHRFEKENRIWTVVLESYVVDMPEGNSEDDTRMLADTVVKLNLQKLATVAEAMARNSGDGSGSQVTFGGGMHGGSAGGSAGGSAGGSSSGSKALLKGVRDFNPISACVCLLENSSDGHSERLFGIGFGPYIIANQHLFRRNNGELTIKTMHLPDQLTEEQIAEYKEAFSLFDKDGDGTITTKELGTVMRSLGHNPTEAELQDMINEVDADGDGTIDFPEFLTMMARKMKYRGGVKLIPSWTTVILVKSMLRKRSFGNPFGGDTEEEIREAFGVFDKDGNGYISAAELRHVMTNLGEKLTDEEVDEMIREADIDGDGQVNYEEFVQMMTAKAAAGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFILWEDAPEDFMGSGASGSGATNFSLLKQAGDVEENPGPGTAIAGFKHASRVTN

SEQ ID NO: 4 - protein, 953 aa, synthetic construct
MGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYKGIDGVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKGSAGSGFEGGELYPYDVPDYAGGLPMSCAQESITSLYKKAGSETVRFQGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGIEEFGLE

SEQ ID NO: 5 - protein, 985 aa, synthetic construct
MGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYKGIDGVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKGSAGSGFEGGELYPYDVPDYAGGLPMSCAQESITSLYKKAGSETVRFQGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGIDGHQDYAEALANPIKHVSLMDQRGSAGSAAGFEEFGLE

SEQ ID NO: 6 - protein, 771 aa, synthetic construct
MGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYGGGQLYPYDVPDYAGGLPMSCAQESITSLYKKAGSETVRFQMGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSAGGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESAGGSAGGSAGGSAGGSAGEFGLE

SEQ ID NO: 7 - protein, 770 aa, synthetic construct
MGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYGGGQLYPYDVPDYAGGLPMSCAQESITSLYKKAGSETVRFQGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSAGGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESAGGSAGGSAGGSAGGSAGEFGLE

SEQ ID NO: 8 - protein, 644 aa, synthetic construct
MGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYGGGQLYPYDVPDYAGGLPMSCAQESITSLYKKAGSETVRFQMGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGIEEFGLE

SEQ ID NO: 9 - protein, 783 aa, synthetic construct
MGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYKGIDGMGGEGDQPLSGYPWFHGMLSRLKAAQLVLTGGTGSHGVFLVRQSETRRGEYVLTFNFQGKAKHLRLSLNEEGQCRVQHLWFQSIFDMLEHFRVHPIPLESGGSSDVVLVSYVPSQRQQGREQAGSGSAGSAAGSGFEELYPYDVPDYAGGLPMSCAQESITSLYKKAGSETVRFQMGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGIEEFGLE

SEQ ID NO: 10 - protein, 787 aa, synthetic construct
MVDIKEKLCYVALDSLEEELDVLVLDDKVAEKLEALSVRGYPYDVPDYAGRVTMGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYGGGQLYPYDVPDYAGGLPMSCAQESITSLYKKAGSETVRFQMGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGIDNTIRVFLPNKQRAVVNVRNGMSLHDCLMKALKVRGLQPECCAVFRLLHEHKGKKARLDWNTDAASLIGEELQVDFLGSAGSAAGSGFEEFGGALE

SEQ ID NO: 11 - protein, 926 aa, synthetic construct
MVDIKEKLCYVALDSLEEELDVLVLDDKVAEKLEALSVRGYPYDVPDYAGRVTMGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYKGIDGMGGEGDQPLSGYPWFHGMLSRLKAAQLVLTGGTGSHGVFLVRQSETRRGEYVLTFNFQGKAKHLRLSLNEEGQCRVQHLWFQSIFDMLEHFRVHPIPLESGGSSDVVLVSYVPSQRQQGREQAGSGSAGSAAGSGFEELYPYDVPDYAGGLPMSCAQESITSLYKKAGSETVRFQMGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGIDNTIRVFLPNKQRAVVNVRNGMSLHDCLMKALKVRGLQPECCAVFRLLHEHKGKKARLDWNTDAASLIGEELQVDFLGSAGSAAGSGFEEFGGALE

SEQ ID NO: 12 - protein, 612 aa, synthetic construct
MVDIKEKLCYVALDSLEEELDVLVLDDKVAEKLEALSVRGGGAGAGGAGGAGGAGAGGAGAGARSLYKGIDGHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPKSYAISFRAEGKIKHCRVQQEGQTVMLGNSEFDSLVDLISYYEKHPLYRKMKLRYPINEEGSAGSAAGFEEFGSAGSAAGSGMHGGSAGGSAGGSAGGSSSGSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFILWEDAPEDFMSGLVPKGVGREYVRFAPGSGASGSGATNFSLLKQAGDVEENPGPGTAIAGFKHASRVGR

SEQ ID NO: 13 - protein, 1003 aa, synthetic construct
MGLFGAPVAGHASGSPAFGTASHSNCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALESGMDELYKAYGGGQLYPYDVPDYAGGLPMSCAQESITSLYKKAGSETVRFQMGCINSKRRDTKLTEEREQKLISEEDLGAPVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKGIDGMMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECGSAGSAAGSGFEEFGLE

SEQ ID NO: 14 - protein, 399 aa, synthetic construct
MKRDHHHHHHQDKKTMMMNEEDDGNGMDELLAVLGYKVRSSEMADVAQKLEQLEVMMSNVQEDDLSQLATETVHYNPAELYTWLDSMLTDLNFGGGMHGGSAGGSAGGSAGGSSSGSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFILWEDAPEDFMSGLVPKGVGREYVRFAPGSGASGSGATNFSLLKQAGDVEENPGPGTAIAGFKHASRVGR

SEQ ID NO: 15 - protein, 879 aa, synthetic construct
MGGRRVRWEVYISRARLVNRQNFVIRLTIGRPGIRRLDPVPRRSAAAIAMLRGQRLGQLGWHRPAAGLGSLMTSLMLACASAASCREVCCPVGPSGLRCTRAGSLDTLRGLRGAGNLTELYVENQQHLQRLEFEDLQGLGELRSLTIVKSGLRFVAPDAFRFTPRLSHLNLSSNALESLSWKTVQGLSLQDLTLSGNPLHCSCALFWLQRWEQEGLCGVHTQTLHDSGPGDQFLPLGHNTSCGVPTVKIQMPNDSVEVGDDVFLQCQVEGLALQQADWILTELEGAATVKKFGDLPSLGLILVNVTSDLNKKNVTCWAENDVGRAEVSVQVSVSFPASVHLGLAVEQHHWCIPFSVDGQPAPSLRWLFNGSVLNETSFIFTQFLESALTNETMRHGCLRLNQPTHVNNGNYTLLAANPYGQAAASVMAAFMDNPFEFNPEDPIPVSFSPVDGNSTSRDPVEKKDETPFGVSVAVGLAVSAALFLSALLLVLNKCGQRSKFGINRPAVLAPEDGLAMSLHFMTLGGSSLSPTEGKGSGLQGHIMENPQYFSDTCVHHIKRQDIILKWELGEGAFGKVFLAECYNLLNDQDKMLVAVAALKEASENARQDFQREAELLTMLQHQHIVRFFGVCTEGGPLLMVFEYMRHGDLNRFLRSHGPDAKLLAGGEDVAPGPLGLGQLLAVASQVAAGMVYLASLHFVHRDLATRNCLVGQGLVVKIGDFGMSRDIYSTDYYRVGGRTMLPIRWMPPESILYRKFSTESDVWSFGVVLWEIFTYGKQPWYQLSNTEAIECITQGRELERPRACPPDVYAIMRGCWQREPQQRLSMKDVHARLQALAQAPPSYLDVLGTRTRPLEQKLISEEDLAANDILDYKDDDDKV

SEQ ID NO: 16 - protein, 612 aa, synthetic construct
MVDIKEKLCYVALDSLEEELDVLVLDDKVAEKLEALSVRGGGAGAGGAGGAGGAGAGGAGAGARSLYKGIDGHSNEKWFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPVPQTNAHESKEWYHASLTRAQAEHMLMRVPRDGAFLVRKRNEPKSYAISFRAEGKIKHCRVQQEGQTVMLGNSEFDSLVDLISYYEKHPLYRKMKLRYPINEEGSAGSAAGFEEFGSAGSAAGSGMHGGSAGGSAGGSAGGSSSGSKALLKGVRDFNPISACVCLLENSSDGHSERLFGIGFGPYIIANQHLFRRNNGELTIKTMHGEFAVANSTQLQMKPVEGRDIIVIKMAKDFPPFPQKLKFRQPTIKDRVCMVSTNFQQKSVSSLVSESSHIVHKEDTSFWQHWITTKDGQAGSPLVSIIDGNILGIHSLTHTTNGSNYFVEFPEKFVATYLDAADGWCKNWKFNADKISWGSFILWEDAPEDFMSGLVPKGVGREYVRFAPGSGASGSGATNFSLLKQAGDVEENPGPGTAIAGFKHASRVGR

SEQ ID NO: 17 - protein, 1003 aa, synthetic construct; identical to SEQ ID NO: 13

SEQ ID NO: 18 - protein, 561 aa, synthetic construct
MWHEGLEEASRLYFGERNVKGMFEVLEPLHAMMERGPQTLKETSFNQAYGRDLMEAQEWCRKYMKSGNVKDLTQAWDLYYHVFRRISKQFGGGMHGGSAGGSAGGSAGGSSSGSKALLKGVRDFNPISACVCLLENSSDGHSERLFGIGFGPYIIANQHLFRRNNGELTIKTMHLPDQLTEEQIAEYKEAFSLFDKDGDGTITTKELGTVMRSLGHNPTEAELQDMINEVDADGDGTIDFPEFLTMMARKMKYRGGVKLIPSWTTVILVKSMLRKRSFGNPFGGDTEEEIREAFGVFDKDGNGYISAAELRHVMTNLGEKLTDEEVDEMIREADIDGDGQVNYEEFVQMMTAKAAAGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFILWEDAPEDFMGSGASGSGATNFSLLKQAGDVEENPGPGTAIAGFKHASRVGR

SEQ ID NO: 19 - protein, 825 aa, synthetic construct
MDVSLCPAKCSFWRIFLLGSVWLDYVGSVLACPANCVCSKTEINCRRPDDGNLFPLLEGQDSGNSNGNASINITDISRNITSIHIENWRGLHTLNAVDMELYTGLQKLTIKNSGLRNIQPRAFAKNPHLRYINLSSNRLTTLSWQLFQTLSLRELRLEQNFFNCSCDIRWMQLWQEQGEARLDSQSLYCISADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLVEPEVRLEHCIEFVVRGNPTPTLHWLYNGQPLRESKIIHMDYYQEGEVSEGCLLFNKPTHYNNGNYTLIAKNALGTANQTINGHFLKEPFPESTDFFDFESDASPTPPITVTHKPEEDTFGVSIAVGLAAFACVLLVVLFIMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 20 - protein, 7 aa, unidentified
EYVRFAP

SEQ ID NO: 21 - protein, 399 aa, synthetic construct
MKRDHHHHHHQDKKTMMMNEEDDGNGMDELLAVLGYKVRSSEMADVAQKLEQLEVMMSNVQEDDLSQLATETVHYNPAELYTWLDSMLTDLNFGGGMHGGSAGGSAGGSAGGSSSGSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFILWEDAPEDFMSGLVPKGVGREYVRFAPGSGASGSGATNFSLLKQAGDVEENPGPGTAIAGFKHASRVTN